(19) United States
(12) Patent Application Publication
(10) Pub. No.: US 2022/0280225 A1
(43) Pub. Date: Sep. 8, 2022

(54) ADJUSTMENT FEATURE FOR ELECTROSURGICAL INSTRUMENT

(71) Applicant: Cilag GmbH International, Zug (CH)

(72) Inventors: Barry C. Worrell, Centerville, OH (US); Gregory A. Trees, Loveland, OH (US); Candice Otrembiak, Loveland, OH (US); Elie R. F. Laumond, South Lebanon, OH (US); Maxwell T. Rockman, Cincinnati, OH (US); Brett M. Elmlinger, Cincinnati, OH (US); John M. Sarley, Mason, OH (US); Roopesh Kumar Manchegowda, Mysore (IN)

(21) Appl. No.: 17/585,702

(22) Filed: Jan. 27, 2022

(30) Foreign Application Priority Data
Mar. 2, 2021 (IN) 202111008714

(51) Int. Cl. A61B 18/14

(52) U.S. Cl. CPC A61B 18/1445; A61B 2018/00208

(57) ABSTRACT

An apparatus includes an end effector, a shaft assembly, a closure jaw assembly, and an adjustment assembly. The end effector includes a first jaw and a second jaw capable to actuate between an open configuration and a closed configuration. The jaw closure assembly includes an elongated jaw closure member, a coupling body attached to the elongated jaw closure member, a yoke housing capable to actuate the coupling body and the elongated jaw closure member to thereby close the second jaw, and an engagement spring capable to bias the coupling body and the elongated jaw closure member into operative engagement with the yoke housing. The adjustment assembly is capable to adjust a spring length of the engagement spring to ensure the coupling body and the elongated jaw closure member remain in operative engagement with the yoke housing.

### PRIORITY

This application claims priority to IN Provisional Pat. App. No. 202111008714, entitled “Adjustment Feature for Electrosurgical Instrument,” filed Mar. 2, 2021.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. Further examples of such devices and related concepts are disclosed in U.S. Pat. No. 6,500,176 entitled “Electrosurgical Systems and Techniques for Sealing Tissue,” issued Dec. 31, 2002, the disclosure of which is incorporated by reference herein; U.S. Pat. No. 8,939,974, entitled “Surgical Instrument Comprising First and Second Drive Systems Actuatable by a Common Trigger Mechanism,” issued Jan. 27, 2015, the disclosure of which is incorporated by reference herein; U.S. Pub. No. 2012/0083783, entitled “Surgical Instrument with Jaw Member,” published Apr. 5, 2012, the disclosure of which is incorporated by reference herein; U.S. Pub. No. 2012/0116379, entitled “Motor Driven Electrosurgical Device with Mechanical and Electrical Feedback,” published May 10, 2012, the disclosure of which is incorporated by reference herein; U.S. Pub. No. 2012/0078243, entitled “Control Features for Articulating Surgical Device,” published Mar. 29, 2012, the disclosure of which is incorporated by reference herein; the disclosure of which is incorporated by reference herein; U.S. Pat. No. 9,545,253, entitled “Surgical Instrument with Contained Dual Helix Actuator Assembly,” issued Jan. 17, 2017, the disclosure of which is incorporated by reference herein; and U.S. Pat. No. 9,526,565, entitled “Electrosurgical Devices,” issued Dec. 27, 2016, the disclosure of which is incorporated by reference herein.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4A depicts a side elevational view of a handle assembly of the electrosurgical instrument of FIG. 1, where the end effector is in an open and unfired state, where a portion of the handle assembly is omitted for purposes of clarity;

FIG. 4B depicts a side elevational view of the handle assembly of FIG. 4A, where the end effector is in a closed and unfired state, where a portion of the handle assembly is omitted for purposes of clarity;

FIG. 4C depicts a side elevational view of the handle assembly of FIG. 4A, where the end effector is in a closed and fired state, where a portion of the handle assembly is omitted for purposes of clarity;

FIG. 5A depicts a cross-sectional side view of the end effector of FIG. 2, where the end effector is in the open and unfired state, taken along line 5-5 of FIG. 2;

FIG. 5B depicts a cross-sectional side view of the end effector of FIG. 2, where the end effector is in the closed and unfired state, taken along line 5-5 of FIG. 2;

FIG. 5C depicts a cross-sectional side view of the end effector of FIG. 2, where the end effector is in the closed and fired state, taken along line 5-5 of FIG. 2;

FIG. 6 depicts a perspective view of a yoke assembly of the handle assembly of FIG. 4A;

FIG. 7 depicts an exploded perspective view of the yoke assembly of FIG. 6;

FIG. 8A depicts a partial cross-sectional view of the handle assembly of FIG. 4A and the yoke assembly of FIG. 6, taken along line 8-8 of FIG. 6, where an engagement spring of the yoke assembly is in a first configuration while the end effector is in the open configuration;

FIG. 8B depicts a partial cross-sectional view of the handle assembly of FIG. 4A and the yoke assembly of FIG. 6, taken along line 8-8 of FIG. 6, where the engagement spring of FIG. 8A is overly compressed as the yoke assembly attempts to proximally translate a jaw closure connector to close the end effector;

FIG. 9 depicts a perspective view of an alternative yoke assembly having an adjustment assembly that may be readily incorporated into the electrosurgical instrument of FIG. 1;

FIG. 10 depicts another perspective view of the yoke assembly of FIG. 9;

FIG. 11 depicts an exploded perspective view of the yoke assembly of FIG. 9;

FIG. 12 depicts a perspective view of a threaded adjustable member of the adjustment assembly of FIG. 9 having a plurality of locking teeth;

FIG. 12A depicts an enlarged perspective view of a plurality of alternative locking teeth that may be readily incorporated into the threaded adjustable member of FIG. 12;

FIG. 13 depicts a perspective view of an adjustable spring seat of the adjustment assembly of FIG. 9 having a plurality of locking teeth;

FIG. 13A depicts a perspective view of an alternative adjustable spring seat having a plurality of alternative locking teeth;

FIG. 14 depicts a perspective view of a yoke housing of the yoke assembly of FIG. 9;

FIG. 15 depicts a sectional view of the yoke housing of FIG. 14, taken along line 15-15 of FIG. 14;

FIG. 16A depicts a cross-sectional view of the yoke assembly of FIG. 9 incorporated into the electrosurgical instrument of FIG. 1, where the adjustment assembly is in a first configuration;

FIG. 16B depicts a cross-sectional view of the yoke assembly of FIG. 9 incorporated into the electrosurgical instrument of FIG. 1, where the adjustment assembly is in a second configuration;

FIG. 17A depicts a top plan view of the adjustment assembly of FIG. 9, where the locking teeth of the threaded adjustment member and the locking teeth of the adjustable spring seat are engaged with each other;

FIG. 17B depicts a top plan view of the adjustment assembly of FIG. 9, where the threaded adjustment member is rotated to compress an engagement spring of the yoke assembly of FIG. 9 such that the locking teeth of the threaded adjustment member and the locking teeth of the adjustable spring seat slip relative to each other;

FIG. 17C depicts a top plan view of the adjustment assembly of FIG. 9, where the threaded adjustment member is further rotated to compress an engagement spring of the yoke assembly of FIG. 9 such that the locking teeth of the threaded adjustment member and the locking teeth of the adjustable spring seat reengage with each other;

FIG. 18 depicts a sectional view of the yoke assembly of FIG. 9, taken along line 18-18 of FIG. 10;

FIG. 19 depicts a cross-sectional view of a second alternative yoke assembly having an adjustment assembly that may be readily incorporated into the electrosurgical instrument of FIG. 1;

FIG. 20 depicts a perspective view of a third alternative yoke assembly having an adjustment assembly that may be readily incorporated into the electrosurgical instrument of FIG. 1;

FIG. 21 depicts a cross-sectional view of the yoke assembly of FIG. 20, taken along line 21-21 of FIG. 20;

FIG. 22 depicts a perspective view of a male threaded member of the adjustment assembly of FIG. 20;

FIG. 23 depicts a perspective view of the female threaded member of the adjustable assembly of FIG. 20;

FIG. 24 depicts a cross-sectional view of the yoke assembly of FIG. 20, taken along line 24-24 of FIG. 20;

FIG. 25 depicts a cross-sectional view of a fourth alternative yoke assembly having an adjustment assembly that may be readily incorporated into the electrosurgical instrument of FIG. 1;

FIG. 26A depicts a cross-sectional view of a fifth alternative yoke assembly having an adjustment assembly that may be readily incorporated into the electrosurgical instrument of FIG. 1, where an engagement spring is in a first configuration;

FIG. 26B depicts a cross-sectional view of the fifth alternative yoke assembly having an adjustment assembly that may be readily incorporated into the electrosurgical instrument of FIG. 1, where the engagement spring of FIG. 26A is in a second configuration;

FIG. 27A depicts a cross-sectional view of a sixth alternative yoke assembly having an adjustment assembly that may be readily incorporated into the electrosurgical instrument of FIG. 1, where an engagement spring is in a first configuration; and

FIG. 27B depicts a cross-sectional view of the sixth alternative yoke assembly having an adjustment assembly that may be readily incorporated into the electrosurgical instrument of FIG. 1, where the engagement spring of FIG. 27A is in a second configuration.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

I. Exemplary Electrosurgical Instrument

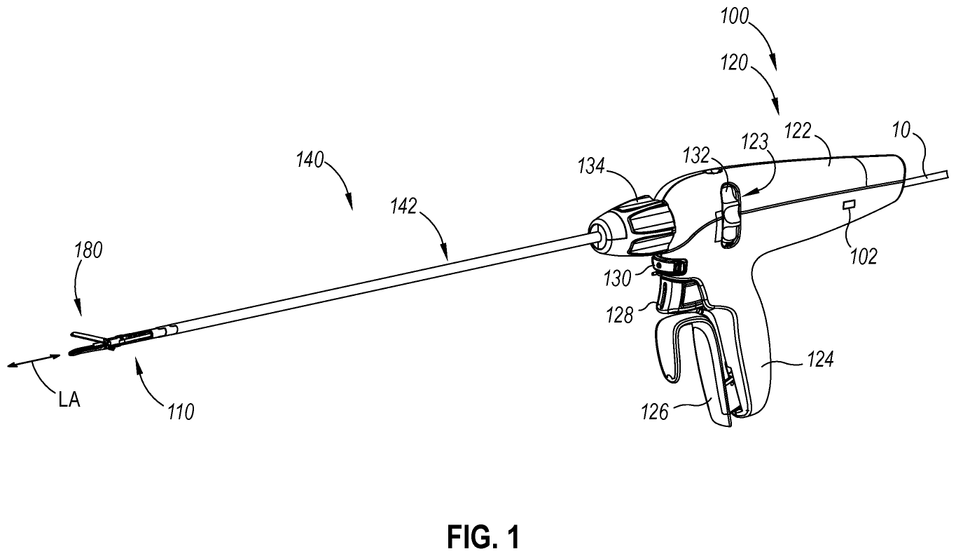

FIGS. 1-3C show an exemplary electrosurgical instrument (100). As best seen in FIG. 1, electrosurgical instrument (100) includes a handle assembly (120), a shaft assembly (140), an articulation assembly (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to seal or weld tissue by applying bipolar radio frequency (RF) energy to tissue. However, it should be understood electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a power source (not shown) via power cable (10).

The power source may be configured to provide all or some of the electrical power requirements for use of electrosurgical instrument (100). Any suitable power source may be used as would be apparent to one skilled in the art in view of the teachings herein. By way of example only, the power source may comprise a GEN04 or GEN11 sold by Ethicon Endo-Surgery, Inc. of Cincinnati, Ohio. In addition, or in the alternative, the power source may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein. While in the current example, electrosurgical instrument (100) is coupled to a power source via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrode surfaces (194, 196) of jaws (182, 184), respectively.

Body (122) of handle assembly (120) defines an opening (123) in which a portion of articulation control (132) protrudes from. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) is configured to bend articulation section (110) in order to drive deflection of end effector (180) from the longitudinal axis (LA) defined by shaft assembly (140). Articulation control (132) and articulation section (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

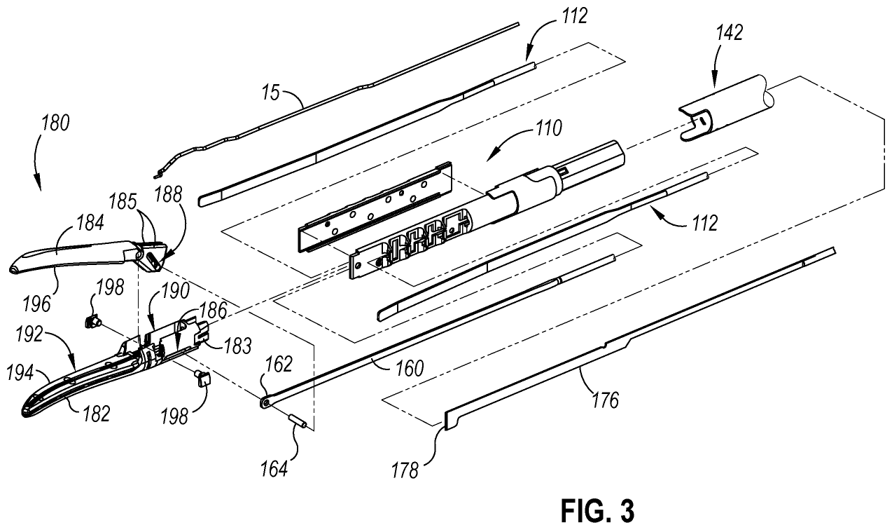

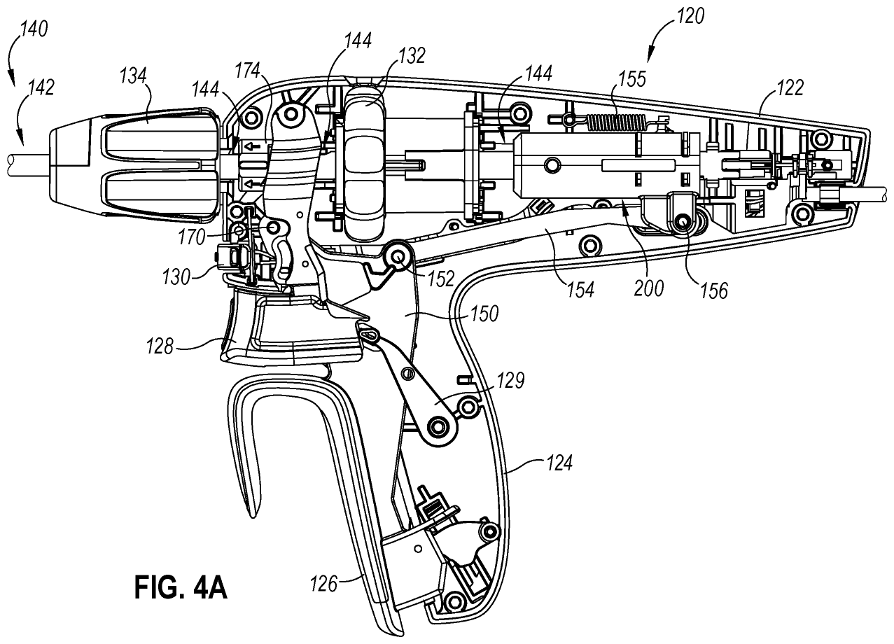

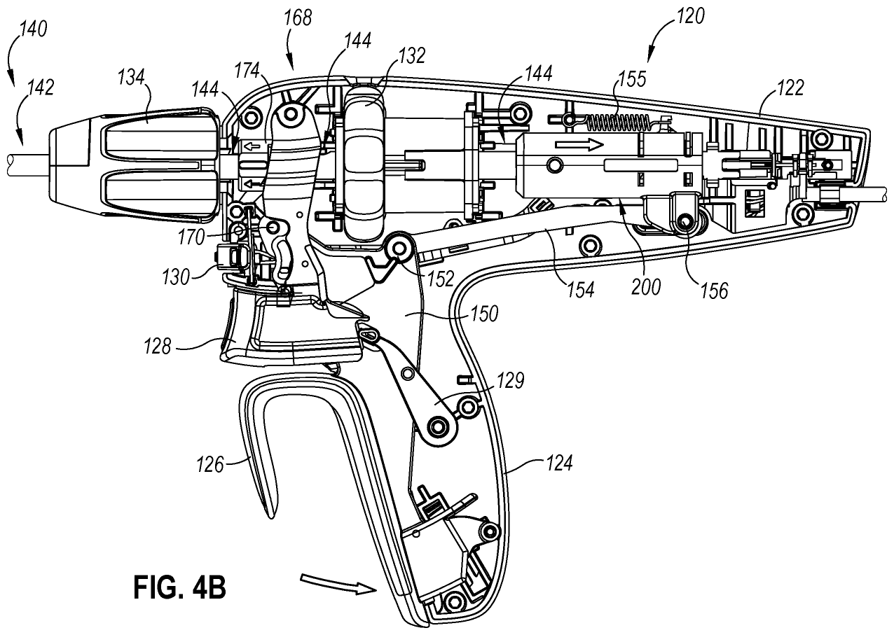

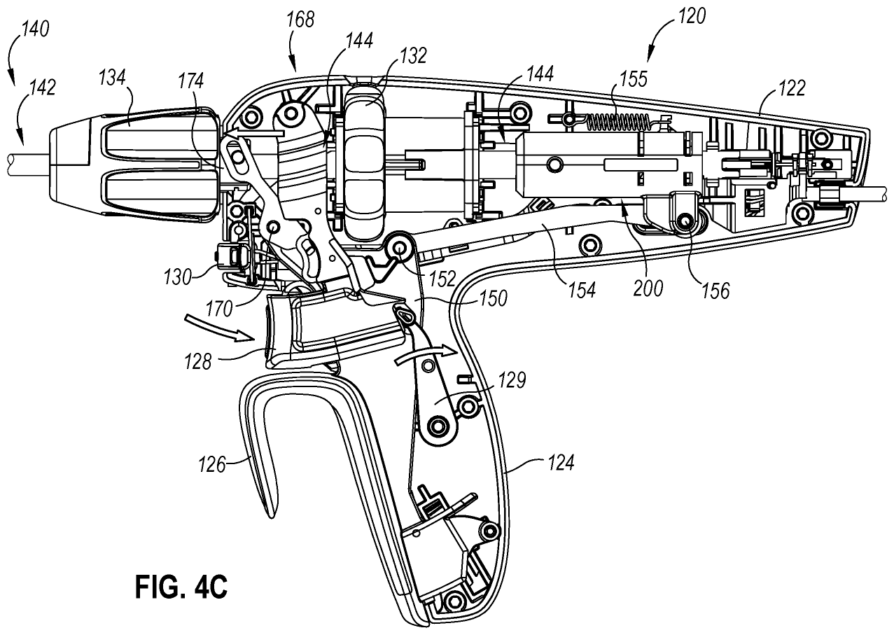

Shaft assembly (140) includes distal portion (142) extending distally from handle assembly (120), and a proximal portion (144) (see FIGS. 4A-4B) housed within the confines of body (122) of handle assembly (120). As best shown in FIG. 3, shaft assembly (140) houses a jaw closure connector (160) that couples jaw closure trigger (126) with end effector (180). Additionally, shaft assembly (140) houses a portion of knife member extending between distal cutting edge (178) and knife trigger (128). Shaft assembly (140) also houses actuating members (112) that couple articulation assembly (110) with articulation control (132); as well as an electrical connecter (15) that operatively couples electrode surfaces (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrode surface (194, 196).

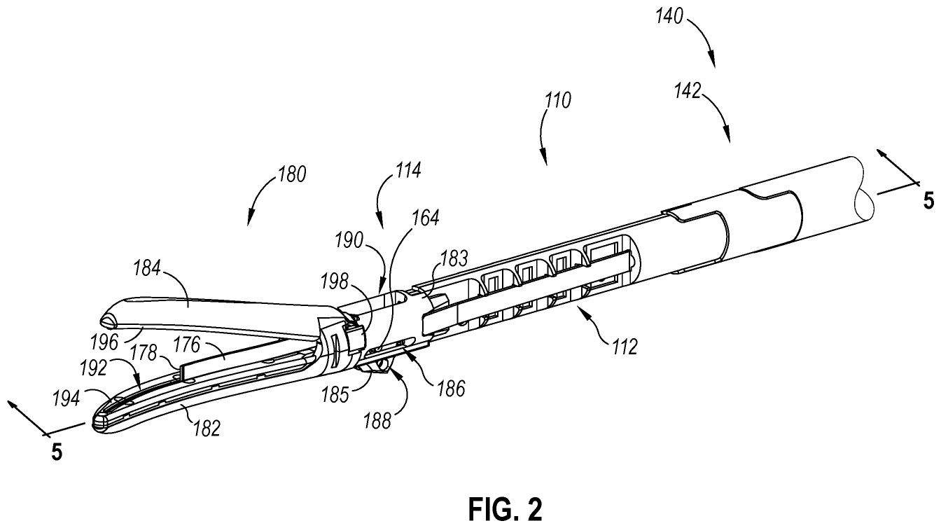

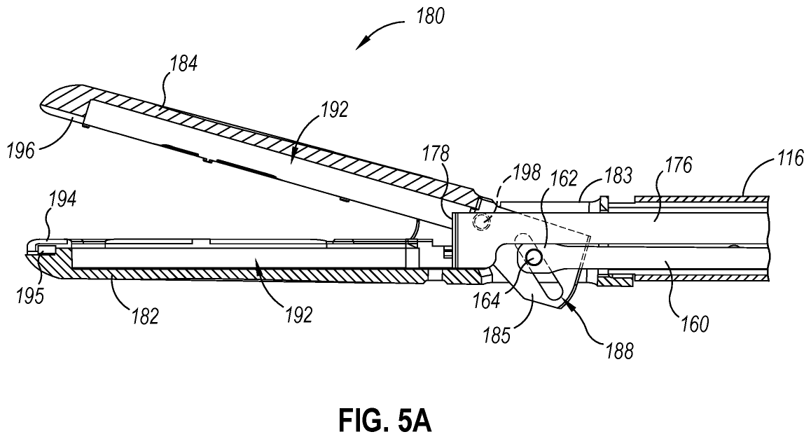

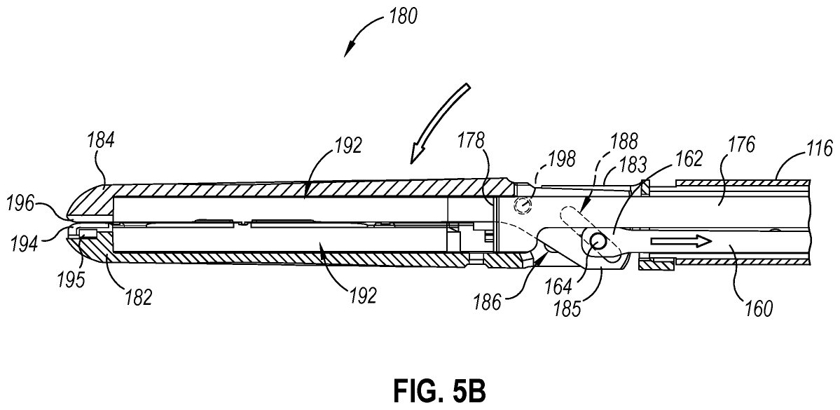

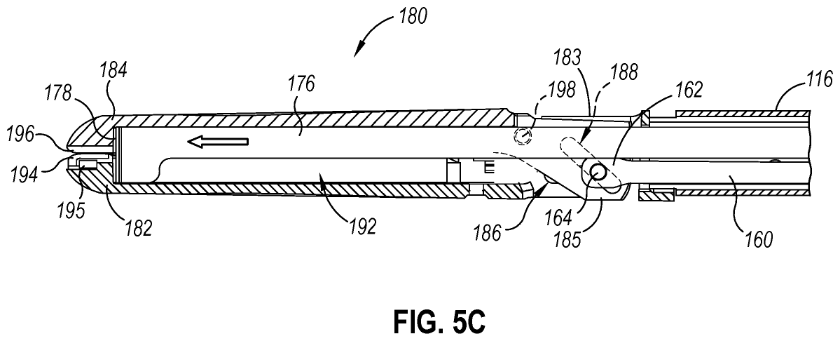

As best seen in FIGS. 2-3, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connecter (160), and pin (164). Slots (186, 188) each slidably receive pin (164), which is attached to a distal coupling portion (162) of jaw closure connector (160). Additionally, as best seen in FIGS. 5A-5C, lower jaw (182) includes a force sensor (195) located at a distal tip of lower jaw (182). Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge.

While in the current example, a force sensor (195) is incorporated into instrument (100) and is in communication with control unit (102), any other suitable sensors or feedback mechanisms may be additionally or alternatively incorporated into instrument (100) while in communication with control unit (102) as would be apparent to one skilled in the art in view of the teachings herein. For instance, an articulation sensor or feedback mechanism may be incorporated into instrument (100), where the articulation sensor communicates signals to control unit (102) indicative of the degree end effector (180) is deflected from the longitudinal axis (LA) by articulation control (132) and articulation section (110).

As will be described in greater detail below, jaw closure connector (160) is operable to translate within central channel (190) of lower jaw (182). Translation of jaw closure connector (160) drives pin (164). As will also be described in greater detail below, because pin (164) is located within both slots (186, 188), and slots (186, 188) are angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis, but may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted (as shown in FIGS. 5A-5B), and advanced (as shown in FIG. 5C), to cut tissue captured between jaws (182, 184). Lower jaw (182) and upper jaw (184) each comprise a respective electrode surface (194, 196). The power source may provide RF energy to electrode surfaces (194, 196) via electrical coupling (15) that extends through handle assembly (120), shaft assembly (140), articulation assembly (110), and electrically couples with one or both of electrode surfaces (194, 196). Electrical coupling (15) may selectively activate electrode surfaces (194, 196) in response to an operator pressing activation button (130). In some instances, control unit (102) may couple electrical coupling (15) with activation button (130), such that control unit (102) activates electrode surfaces (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc.

FIGS. 4A-5C show an exemplary use of instrument (100) for end effector (180) to grasp, cut, and seal/weld tissue. As described above, and as shown between FIGS. 4A-4B and 5A-5B, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

Handle assembly (120) further includes a yoke assembly (200) that is slidably coupled along proximal portion (144) of shaft assembly (140). Yoke assembly (200) is operatively coupled with jaw closure connector (160) such that translation of yoke assembly (200) relative to proximal portion (144) of shaft assembly (140) translates jaw closure connector (160) relative to shaft assembly (140).

As best seen in FIGS. 4A-4C, yoke assembly (200) is coupled to a body (150) of jaw closure trigger (126) via a link (154). Link (154) is pivotally coupled with yoke assembly (200) via pin (156); while link (154) is also pivotally coupled with body (150) of jaw closure trigger (126) via pin (152). Additionally, jaw closure trigger (126) is pivotally coupled with body (122) of handle assembly (120) via pin (170). Therefore, as shown between FIGS. 4A-4B, an operator may pull jaw closure trigger (126) toward pistol grip (124), thereby rotating jaw closure trigger (126) about pin (170). Rotation of jaw closure trigger (126) leads to rotation of link (154) about both pins (152, 156), which in turn drives yoke assembly (200) in the proximal direction along proximal portion (144) of shaft assembly (140).

As described above, jaw closure connector (160) extends within shaft assembly (140), articulation section (110), and central channel (190) of lower jaw (182). As also mentioned above, jaw closure connector (160) is attached to pin (164). Therefore, as seen between FIGS. 5A-5B, proximal translation of yoke assembly (200) leads to proximal translation of pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

As best seen in FIGS. 4A-4C, yoke assembly (200) is also coupled with a bias spring (155). Bias spring (155) is also coupled to a portion of body (122), such that bias spring (155) biases yoke assembly (200) to the position shown in FIG. 4A (associated with the open configuration of end effector (180) as shown in FIG. 5A). Therefore, if an operator releases jaw closure trigger (126), bias spring (155) will translate yoke assembly (200) to the position shown in FIG. 4A, thereby opening jaws (182, 184) of end effector (180).

As described above, and as shown between FIGS. 4B-4C and 5B-5C, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body (174) that is slidably coupled along proximal portion (144) of shaft assembly (140). Knife coupling body (174) is coupled with knife member (176) such that translation of knife coupling body (174) relative to proximal portion (144) of shaft assembly (140) translates knife member (176) relative to shaft assembly (140).

As best seen in FIGS. 4B-4C and 5B-5C, knife coupling body (174) is coupled a knife actuation assembly (168) such that as knife trigger (128) pivots toward body (122) and/or pistol grip (124), knife actuation assembly (168) drives knife coupling body (174) distally, thereby driving knife member (176) distally within knife pathway (192). Because knife coupling body (174) is coupled to knife member (176), knife member (176) translates distally within shaft assembly (140), articulation section (110), and within knife pathway (192) of end effector (180), as best shown between FIGS. 5B-5C. Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

Knife trigger (128) is biased to the positions seen in FIGS. 4A-4B (associated with the knife member (176) in the retracted position) by a bias arm (129). Bias arm (129) may include any suitable biasing mechanism as would be apparent to one having ordinary skill in the art in view of the teachings herein. For instance, bias arm (129) may include a torsion spring. Therefore, if an operator releases knife trigger (128), bias arm (129) returns knife trigger (128) to the position shown in FIGS. 4A-4B, thereby translating knife member (176) toward the retracted position.

With distal cutting edge (178) of knife member (176) actuated to the advance position (position shown in FIG. 5C), an operator may press activation button (130) to selectively activate electrode surfaces (194, 196) of jaws (182, 184) to weld/seal severed tissue that is captured between jaws (182, 184). It should be understood that the operator may also press activation button (130) to selectively activate electrode surfaces (194, 196) of jaws (182, 184) at any suitable time during exemplary use. Therefore, the operator may also press activation button (130) while knife member (176) is retracted as shown in FIGS. 3A-3B. Next, the operator may release jaw closure trigger (128) such that jaws (182, 184) pivot into the opened configuration, releasing tissue.

II. Exemplary Yoke Assembly

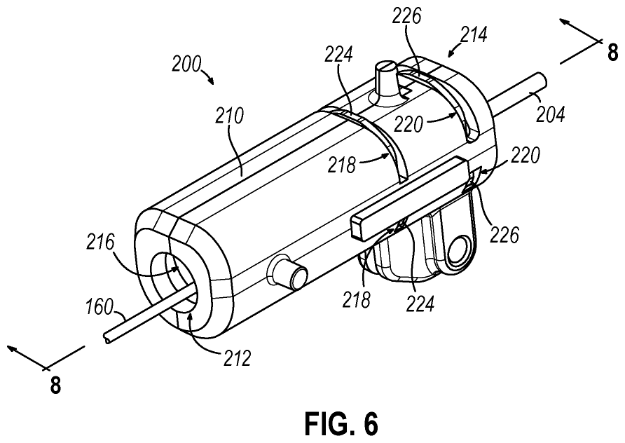

FIGS. 6-8B further show yoke assembly (200). As mentioned above, yoke assembly (200) is coupled to elongated jaw closure member (160) such that actuation of yoke assembly (200) relative to shaft assembly (140) may drive actuation of elongated jaw closure member (160) relative to shaft assembly (140), thereby closing and opening jaws (182, 184) in accordance with description herein.

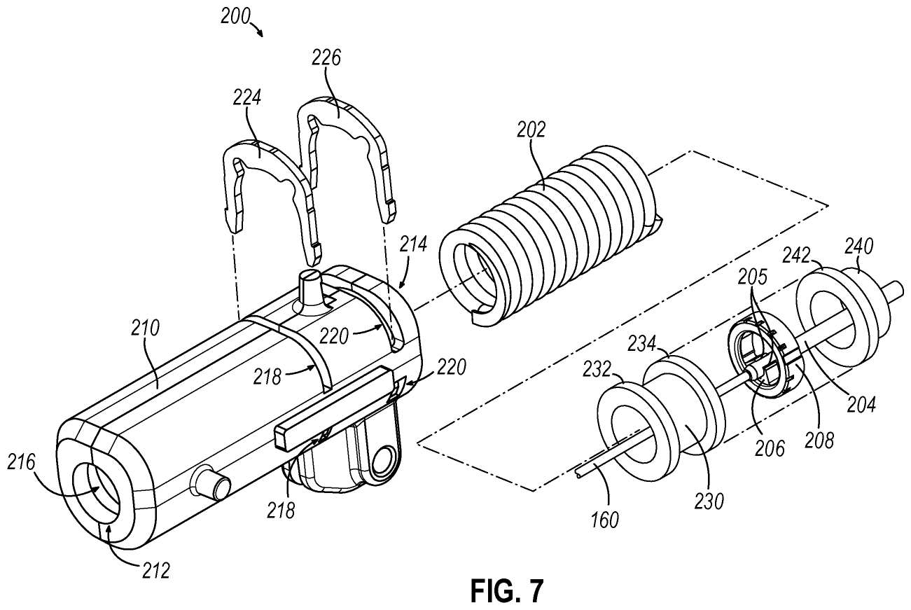

As best shown in FIG. 7, yoke assembly (200) includes a yoke housing (210), an engagement spring (202), a hollow pull tube (204), a rotating connecting body (206), a translating ring (208), a spring engagement collar (230), a proximal collar (240), a first clip (224), and a second clip (226).

Yoke housing (210) defines an internal cavity (216) extending between an open distal end (212) and an open proximal end (214). Internal cavity (216) houses engagement spring (202), collars (230, 240), jaw closure member (160), hollow pull tube (204), rotating connection body (206), and translating ring (208); while clips (224, 226) are used to ensure the housed components remain operatively housed within internal cavity (216). While not shown for purposes on clarity, proximal portion (144) of shaft assembly (140), we well as various component housed within shaft assembly (140), may also extend through yoke housing (210), engagement spring (202), collars (230, 240), and translating ring (208).

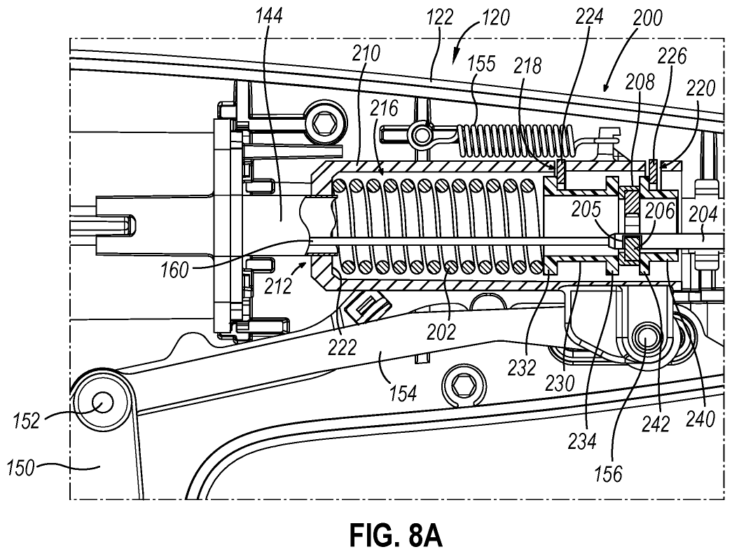

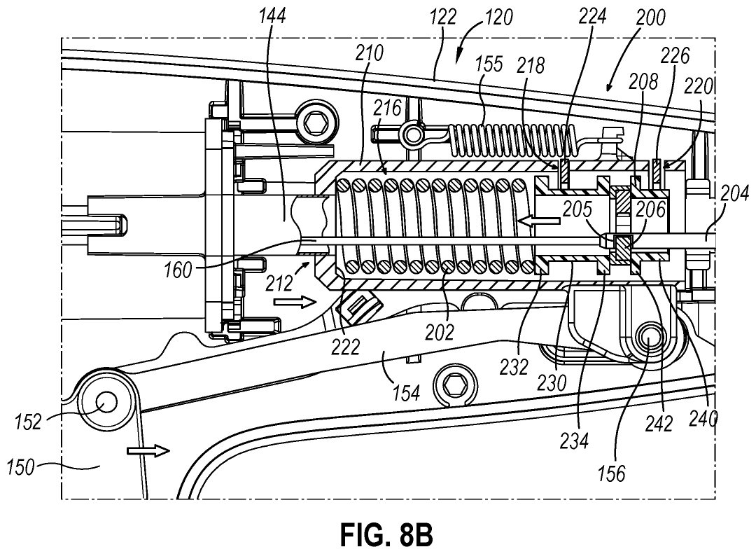

As best shown in FIGS. 8A-8B, hollow pull tube (204) is coupled to rotating connecting body (206) via a pair of flats (205) such that hollow pull tube (204) rotates and translates with rotating connecting body (206). Hollow pull tube (204) is coupled with elongated jaw closure member (160) such that hollow pull tube (204) is fixed to elongated jaw closure member (160) when fully assembled.

Rotating connecting body (206) is rotatably disposed within translating ring (208) such that rotating connecting body (206), hollow pull tube (204), and elongated jaw closure member (160) may rotate with shaft assembly (140) about the longitudinal axis (LA) of shaft assembly (140) relative to the rest of yoke assembly (200). However, rotating connection body (206) is configured to translate with translating ring (208) in accordance with the description herein.

It should be understood that elongated jaw closure member (160) and at least a portion of hollow pull tube (204) are housed within shaft assembly (140), while proximal portion (144) of shaft assembly (140) extends through translating ring (208). Therefore, a section of proximal portion (144) adjacent to translating ring (208) may define a slot dimensioned to accommodate translation of rotating connecting body (206), hollow pull tube (204), and elongated jaw closure member (160) in order for elongated jaw closure member (160) to translate relative to shaft assembly (140) to thereby acuate jaws (182, 184) in accordance with the description herein.

As best shown in FIG. 8A, when fully assembled, a distal end of engagement spring (202) abuts against a proximally facing interior surface (222) of yoke housing (210), while a proximal end of engagement spring (202) abuts against a spring side flange (232) of spring engagement collar (230). Engagement spring (202) is configured to abut against spring engagement collar (230) with suitable force such that second flange (234) of spring engagement collar (230) is in contact with translating ring (208). Proximal collar (240) and clip (226) are configured to keep translating ring (208) from being pushed out of cavity (216) by the force of engagement spring (202).

Therefore, engagement spring (202) is configured to maintain contact with spring engagement collar (230) with a suitable force such that spring engagement collar (230) and translating ring (208) remains in a substantially fixed longitudinal position relative to yoke housing (210). Therefore, when yoke housing (210) is driven proximally via closure of trigger (126), engagement spring (202) forces translating ring (208), rotating connecting body (206), hollow pull tube (204), and elongated jaw closure member (160) to also proximally translate around the same distance as yoke housing (210) in order to suitably close jaws (182, 184) in accordance with the description herein. Conversely, when yoke housing (210) is driven distally via opening of trigger (126); clip (226) and proximal collar (240) drive translating ring (208), rotating connecting body (206), hollow pull tube (204) and elongated jaw closure member (160) distally the around the same distance as yoke housing (210) in order to suitable open jaws (182, 184) in accordance with the description herein.

Clips (224, 226) are configured to be selectively inserted into respective clip slots (218, 220) during assembly to thereby engage respective flanges (232, 242) of respective collars (230, 240) and prevent internal components of yoke assembly (200) from distally exiting cavity (216) of yoke housing (210). Clips (224, 226) may be inserted during any suitable time during the assembly process as would be apparent to one skilled in the art in view of the teachings herein.

III. Exemplary Alternative Yoke Assemblies with Adjustment Feature

As mentioned above, an operator may selectively activate electrode surfaces (194, 196) of jaws (182, 184) to weld/seal tissue that is captured between jaws (182, 184) in accordance with the description above. In order to help ensure a quality weld/seal of grasped tissue using RF energy, it may be desirable to ensure that jaws (182, 184) grasp tissue with a suitable closure force. If the closure force is too low, electrode surfaces (194, 196) of jaws (182, 184) may not be able to apply a suitable degree of RF energy to the tissue to thereby achieve the desired degree of welding or sealing to the tissue. If the closure force is too high, jaws (182, 184) may over-compress the tissue and thereby cause structural damage to the tissue, such that the tissue is unable to form a suitable weld or seal even if the appropriate amount of RF energy is applied to the tissue. As also mentioned above, and as shown in FIGS. 5A-5B, elongated jaw closure member (160) may be actuated proximally such that pin (164) cams against slots (186, 188) in order to pivot jaws (182, 184) about pivot couplings (198) to grasp tissue with the above mentioned suitable closure force. In other words, the proximal force imparted on elongated jaw closure member (160) is utilized to generate the suitable closure force required for jaws (182, 184) and electrode surfaces (194, 196) to provide quality weld/seal of grasped tissue.

During assembly of yoke assembly (200), an operator or manufacturing machine may pull on a proximal end of elongated jaw closure member (160) prior to jaw closure member (160) being fixed to hollow pull tube (204), but still being slidably contained within hollow pull tube (204). In particular, an operator or manufacturing machine may pull proximal end of elongated jaw closure member (160) with sufficient proximal force required for elongated jaw closure member (160) to suitably close jaws (182, 184) in accordance with the description herein. It should be understood that during this assembly process, the external force pulling on elongated jaw closure member (160) is generating the closure force on jaws (182, 184), rather than the yoke assembly (200), link (154), and trigger (126).

While pulling on the proximal end of elongated jaw closure member (160) with an external force, an operator or manufacturing machine may place other suitable components of yoke assembly (200) in the longitudinal position associated with jaws (182, 184) being fully closed during exemplary operation. Once the suitable closure force is measured between jaws (182, 184), an operator or manufacturing machine may then fix elongated jaw closure member (160) to hollow pull tube (204) via any suitable technique as would be apparent to one skilled in the art in view of the teachings herein, such as welding.

Due to the accumulation of various tolerances while manufacturing and assembling components of instrument (100), a first assembled instrument (100) may require a first proximal force required for elongated jaw closure member (160) to suitably close jaws (182, 184), while a second assembled instrument (100) may require a second, different, proximal force required for elongated jaw closure member (160) to suitably close jaws (182, 184). For instance, the friction between pin (164) and both slots (186, 188) may deviate between assembled instruments (100) due to a variety of reasons. As another example, the location of pivot couplings (198) may deviate between assembled instruments (100) due to a variety of reasons. The deviations may lead to a difference in proximal force required for one elongated jaw closure member (160) of a first instrument (100) to suitably close jaws (182, 184) as compared to another elongated jaw closure member (160) of a second instrument (100).

Additionally, various tolerances of engagement spring (202) may exist, such as quality of material, length of spring (202), spring constant, etc.; which may affect the maximum proximal force engagement spring (202) may impart on spring engagement collar (230) and translating ring (208) without spring (202) undesirably compressing. As mentioned above, engagement spring (202) forces translating ring (208), rotating connecting body (206), hollow pull tube (204), and elongated jaw closure member (160) to proximally translate with yoke housing (210). In other words, once assembled, engagement spring (202) may help impart the suitable proximal force onto elongated jaw closure member (160) such that elongated jaw closure member (160) may suitably close jaws (182, 184) in accordance with the description herein.

If a tolerance stack accumulates to a degree such that the proximal force required for elongated jaw closure member (160) to suitably close jaws (182, 184) is larger than the force engagement spring (202) is capable of imparting on spring engagement collar (230) without undesirably compressing, an operator may not be able to suitably close jaws (182, 184) in accordance with the description herein. In such an instance, as depicted between FIGS. 8A-8B, a user may pull trigger (126) expecting to suitably close jaws (182, 184). However, since the proximal force needed to suitable close jaws (182, 184) is larger than the force provided by engagement spring (202), engagement spring (202) may undesirably compress, leading to an undesirable amount of relative translation between yoke housing (210) and translating ring (208) (and therefore jaw closure member (160)). With elongated jaw closure member (160) not proximally actuated into the desired proximal position, pin (150) may not proximally acuate within slots (186, 188) to suitably close jaws (182, 184). Therefore, in some instances, even if the operator pulls trigger (126) to the position the operator believes to be associated with jaws (182, 184) being suitably closed, jaws (182, 184) may not be able to reach such a position.

Therefore, it may be desirable to provide a yoke assembly (200) configured to tune or adjust the length of engagement spring (202) after elongated jaw closure member (160) is fixed to hollow pull tube (204). Such tuning or adjusting may ensure engagement spring (202) in providing a sufficient reactionary force to prevent an understandable amount of relative translation (caused by undesirable compression of spring (202)) between elongated jaw closure member (160) and yoke housing (210) during exemplary closing of jaws (182, 184).

A. First Exemplary Yoke Assembly with Adjustment Feature

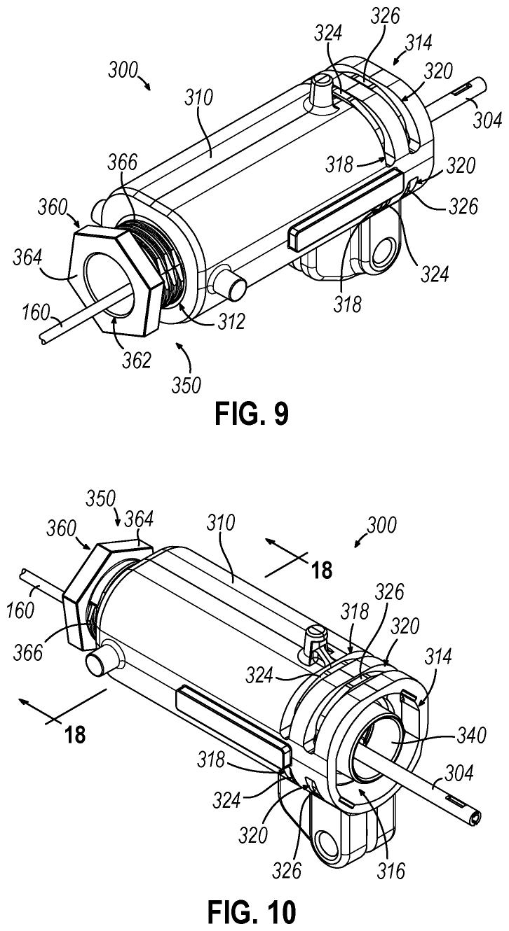

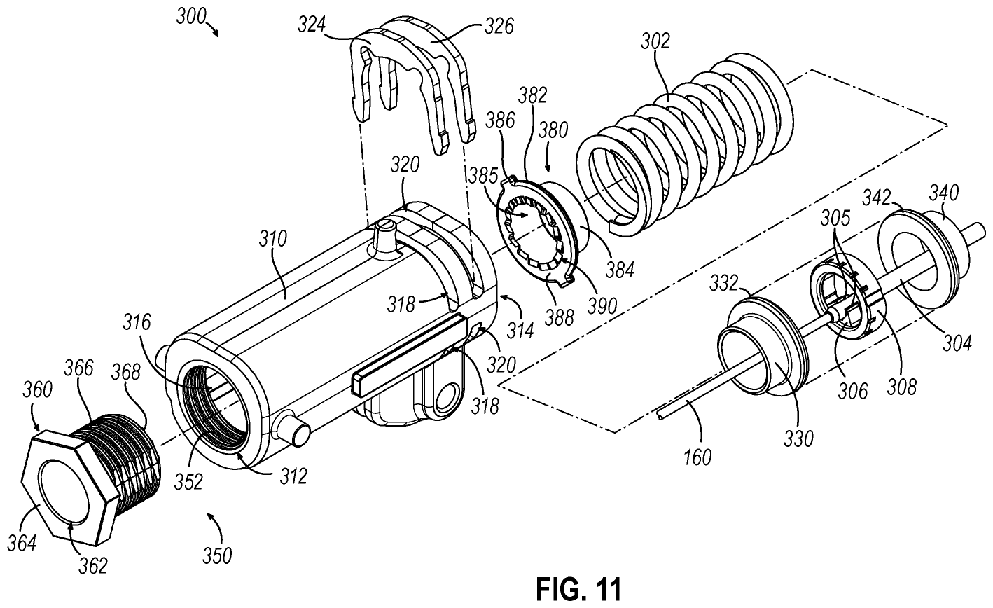

FIGS. 9-11 show an exemplary yoke assembly (300) that may be readily incorporated into instrument (100) in replacement of yoke assembly (200) described above. Yoke assembly (300) is substantially similar to yoke assembly (200) described above, with differences elaborated below. In particular, yoke assembly (300) includes an adjustment assembly (350) configured to adjust or tune the length of an engagement spring (302) after elongated jaw closure member (160) is fixed to a hollow pull tube (304).

Therefore, yoke assembly (300) includes engagement spring (302), hollow pull tube (304) having flat surfaces (305), a rotating connecting body (306), a translating ring (308), a yoke housing (310), a first clip (324), a second clip (326), a spring engagement collar (330), and a proximal collar (340) having a flange (342); which are substantially similar to engagement spring (202), hollow pull tube (204) having flat surfaces (205), rotating connecting body (206), translating ring (208), yoke housing (210), first clip (224), second clip (226), spring engagement collar (230), and proximal collar (240) having flange (242), respectively, with differences elaborated below.

Yoke housing (310) defines an open distal end (312), an open proximal end (314), a cavity (316), and clip slots (318, 320); which are substantially similar to open distal end (212), open proximal end (214), cavity (216), and clip slots (218, 220), respectively described above, with difference elaborated below.

Spring engagement collar (330) in the current example has a single flange (332) configured to abut against both engagement spring (302) and translating ring (308). Additionally, spring engagement collar (330) includes a distally extending sleeve dimensioned to be housed within engagement spring (302).

Adjustment assembly (350) includes a female threading (352) associated with open distal end (312) of yoke housing (310), a threaded adjustable member (360), and an adjustable spring seat (380). As will be described in greater detail below, threaded adjustable member (360) includes a threaded body (366) that suitably meshes with female threading (352) of yoke housing (310) such that an operator or manufacturing machine may change the longitudinal position of threaded adjustable member (360) relative to yoke housing (310), and therefore selectively adjust/tune the spring length (and therefore the preload force) of engagement spring (302) during assembly of instrument (100). Therefore, an operator or manufacturing machine may change the amount of proximal force engagement spring (302) imparts on collar (330) and translating ring (308) in order to inhibit undesirable spring compressing during exemplary closure of jaws (182, 184) in accordance with the description herein.

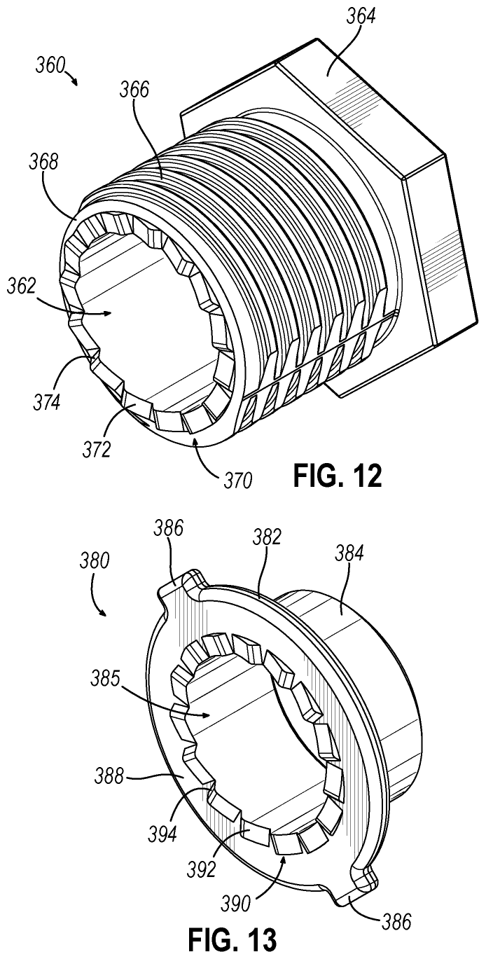

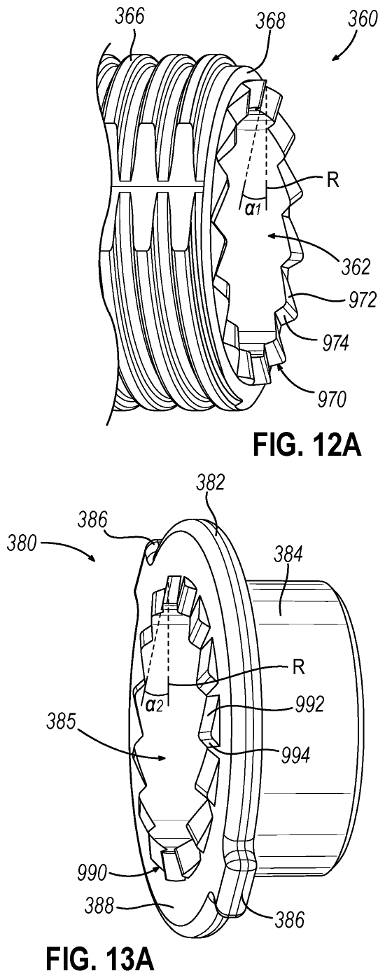

As best shown in FIG. 12, threaded adjustable member (360) includes a head (364), threaded body (366) terminating into a proximal face (368), and a plurality of locking teeth (370). Threaded adjustable member (360) also defines a through hole (362) extending from a proximal end to a distal end of threaded adjustable member (360). Through hole (362) is dimensioned to receive selective portions of shaft assembly (140).

As mentioned above, threaded body (360) is suitably engaged with female threading (352) of yolk housing (310). Head (364) is dimensioned to be grasped by an assembler or a tool in order to rotate threaded adjustable member (360) relative to yoke housing (310). Due to threaded body (366) suitably meshing with female threading (352), rotation of threaded adjustable member (360) relative to yoke housing (310) alters the longitudinal position of threaded adjustable member (360) relative to yoke housing (310). In particular, rotation of threaded body (360) in a first rotational direction actuates threading body (360) proximally toward cavity (316); while rotation of threaded body (360) in a second rotational direction actuates threaded body (360) distally away from cavity (316).

As will be described in greater detail below, proximal face (368) of threaded adjustable member (360) abuts against a distal face (388) of adjustable spring seat (388) such that the longitudinal position of adjustable member (360) determines the longitudinal position of adjustable spring seat (380) relative to yoke housing (310). As will also be described in greater detail below, adjustable spring seat (380) engages a distal end of engagement spring (302) such that the longitudinal position of adjustable spring seat (380) determines the spring length of engagement spring (302).

As best shown in FIG. 13, adjustable spring seat (380) includes a spring engagement flange (382), an interior spring sleeve (384) extending proximally from spring engagement flange (382), a pair of angular locking protrusions (386) extending laterally from flange (382), distal face (388), and a plurality of locking teeth (390). Adjustable spring seat (380) also defines a through hole (385) extending from a proximal end to a distal end of adjustable spring seat (380). Through hole (385) is dimensioned to receive selective portions of shaft assembly (140).

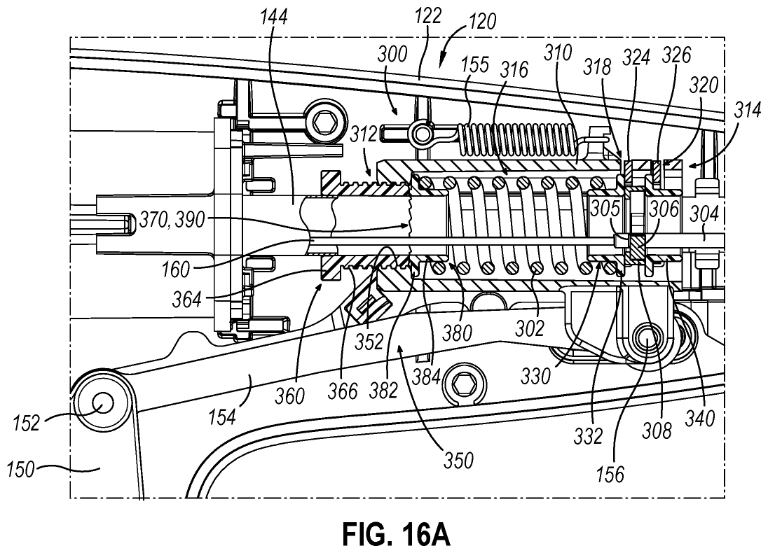

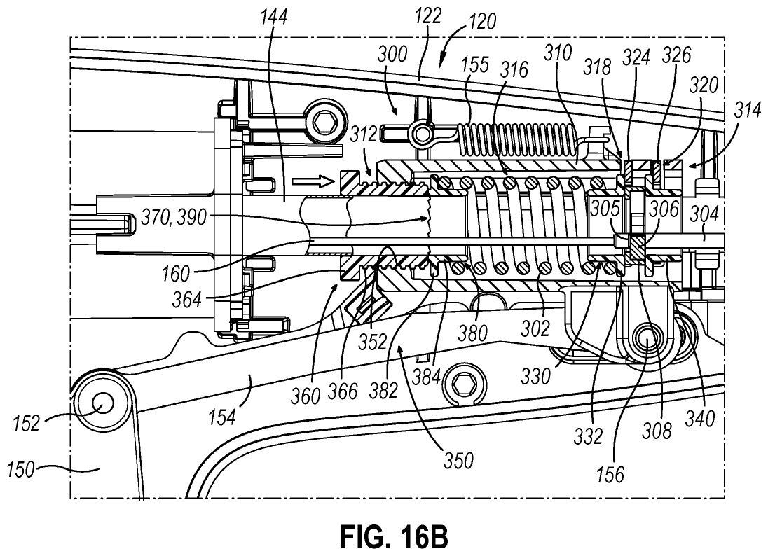

FIGS. 16A-16B show exemplary use of adjustable assembly (350) in order to change the spring length (and therefore the preload force) of engagement spring (302), to thereby increase the proximal reactionary force (i.e. the preload force) engagement spring (302) can impart on spring engagement collar (330) during exemplary closing of jaws (182, 184) in accordance with the description herein. FIG. 16A shows threaded adjustable member (360) in a distal position such that spring engagement flange (382) abuts against a proximally facing interior wall of yoke housing (310). Spring engagement flange (382) abuts against the distal end of engagement spring (302), while distal face (388) abuts against proximal face (368) of threaded adjustable member (360). In the position shown in FIG. 16A, engagement spring (302) may have a spring length too great in order to apply a suitable reaction force to elongated jaw closure member (160) during exemplary closing of jaws (182, 184).

Therefore, as shown in FIG. 16B, an assembler may desire to further compress engagement spring (302) by rotating threaded adjustable member (360) in the first rotational direction in accordance with the description herein. Proximal face (368) translates proximally into cavity (316) in response to rotation of threaded adjustable member (360) in the first direction. Since distal face (388) of spring engagement flange (382) abuts against proximal face (368) of threaded adjustable member (360), adjustable spring seat (380) is driven proximally within cavity (316) as well. Since a distal end of engagement spring (302) abuts against engagement flange (382), engagement spring (302) effectively compresses against flanges (382, 332), shortening the spring length of engagement spring (302). With a shorter spring length, engagement spring (302) may impart a greater proximal reactionary force (i.e. a preload force) against spring engagement collar (330) without undesirably compressing during elementary closing of jaws (182, 184). In other words, adjustment assembly (350) may be utilized to ensure closure of trigger (126) results in a suitable closure force between jaws (182, 184).

Interior spring sleeve (384) is housed within the interior of engagement spring (302). Interior spring sleeve (384), as well as the portion of spring engagement collar (330) within the interior of engagement spring (302), may help ensure engagement spring (302) retains structural integrity (i.e. a central portion of spring (302) does not buckle) as the length of engagement spring (302) is changed in accordance with the description herein.

In some instances, it may be desirable to know how far engagement spring (302) has been compressed. Additionally or alternatively, in some instances, after adjustment assembly (350) is utilized to compress spring (302) into a desired spring length, it may be desirable to ensure that threaded adjustable member (360) does not accidentally rotate in the second angular direction associated with distal movement of threaded body (366) and therefore allow spring (302) to undesirably increase in spring length.

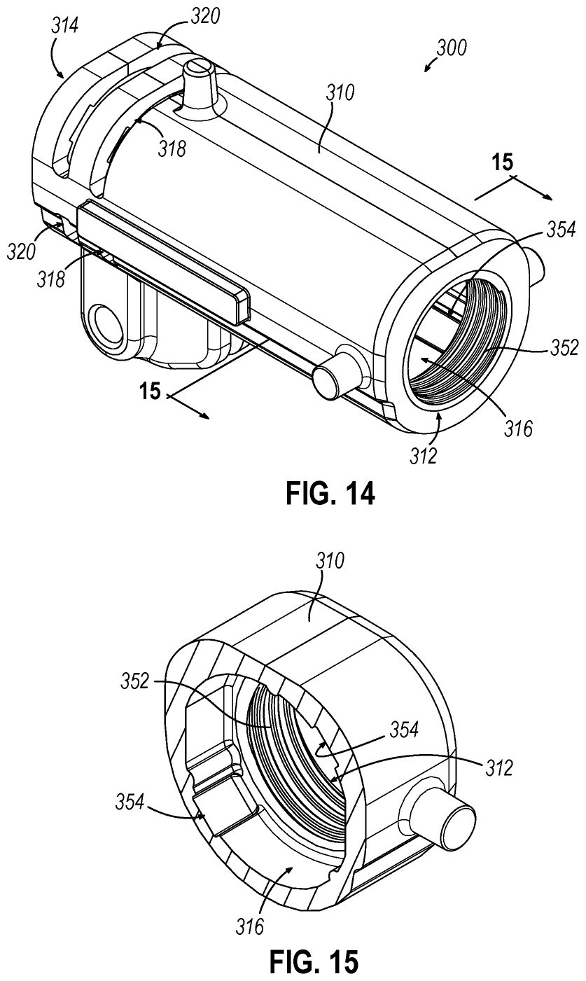

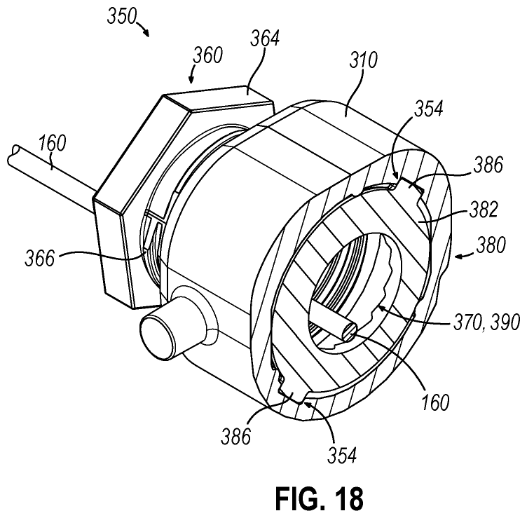

As shown in FIGS. 14-15, an interior surface of yoke housing (310) defining cavity (316) also defines two longitudinally extending channels (354). As mentioned above, adjustable spring seat (380) includes a pair of angular locking protrusion (386). As best shown in FIG. 18, longitudinal extending channels (354) are dimensioned to receive angular locking protrusions (386) such that spring seat (380) may translate within cavity (316) in order to compress spring (302), but also such that spring seat (380) is rotationally fixed about longitudinal axis (LA) relative to yoke housing (310).

As mentioned above, and as shown in FIGS. 12-13, threaded adjustable member (360) and adjustable spring seat (380) both include a plurality of locking teeth (370, 390). As will be described in greater detail below, locking teeth (368) of threaded adjustable member (360) are configured to mesh with locking teeth (390) of adjustable spring seat (380) in order to inhibit engagement spring (302) from increasing spring length after assembly.

Locking teeth (370, 390) are disposed on faces (368, 388) in an annular array. Each tooth (370, 390) includes a gradually sloped surface (372, 392) and an inclined sloped surface (374, 394). Locking teeth (370, 390) are arranged to mesh with each other such that as threaded body (366) is rotated in the first angular direction associated with compressing engagement spring (302), gradually sloped surfaces (372, 392) cam against each other.

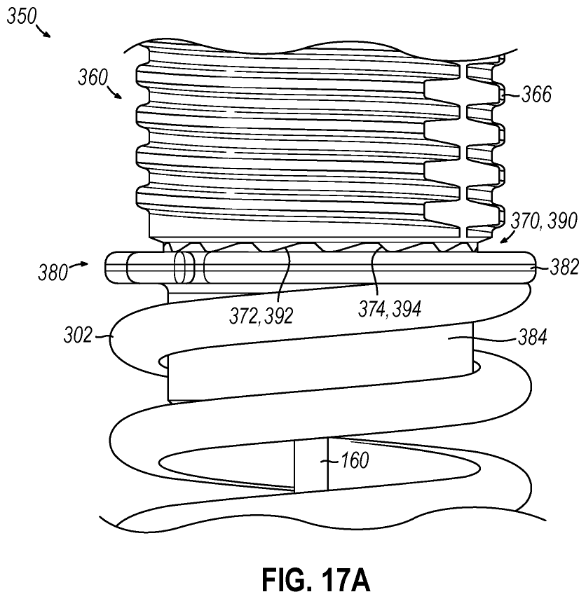

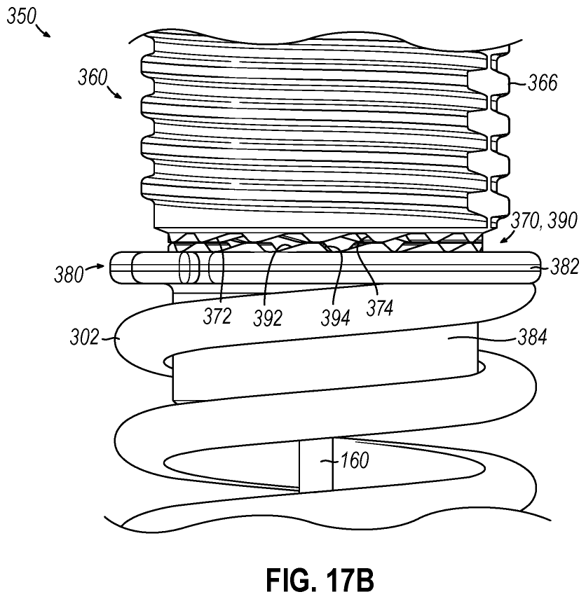

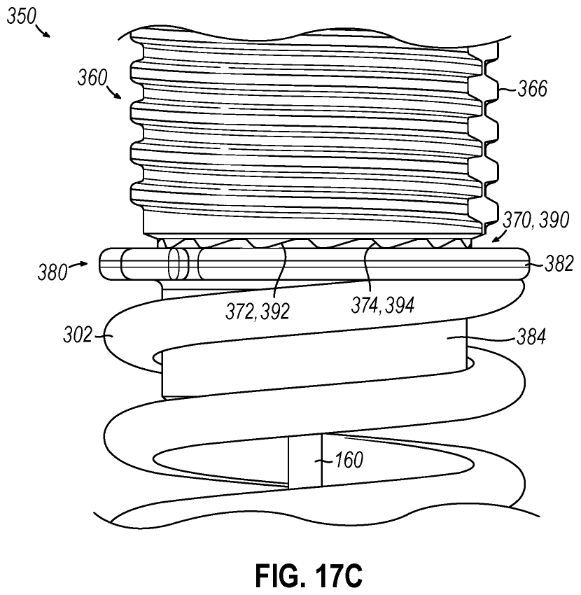

FIGS. 17A-17C show rotation of threaded body (366) in the first angular direction such that locking teeth (370, 390) traverse each other on gradually sloped surfaces (372, 392. As shown between FIGS. 17A-17B, as teeth (370, 390) traverse each other, spring seat (380) may temporarily extend away from proximal face (368) of threaded body (366). Once locking teeth (370, 390) finish traversing each other, as shown between FIG. 17B-17C, spring (302) may acuate spring seat (380) back toward proximal face (368) of threaded body (366). This actuation of spring seat (380) back toward proximal face (368) may provide tactile and/or audible feedback to the user that locking teeth (370, 390) finished traversing each other, indicating to the user threaded body (366) rotate relative to both spring seat (380) and yoke housing (310) a certain rotational amount. Additionally, since spring seat (380) is rotationally fixed relative to yoke housing (310), locking teeth (370, 390) may be disposed in such a manner that each tactile and/or audible feedback may represent a specific length which engagement spring (302) has been compressed

Conversely, locking teeth (370, 390) are arranged to mesh with each other such that as threaded body (366) is rotated in the second angular direction associated with lengthening engagement spring (302), inclined sloped surfaces (374, 394) cam against each other. The geometry of inclined sloped surfaces (374, 394) may require a larger amount of torque in order to rotate threaded body (366) in the second angular direction as compared to if there were no locking teeth (370, 390). Inclined sloped surfaces (374, 394) may have other characteristics to help increase the torque required to rotate threaded body (366) in the second angular direction. For instance, inclined sloped surfaces (374, 394) may have a roughened surface, a larger coefficient of friction, or any other suitable feature that would increase to torque required to rotate threaded body (366) in the second angular direction as would be apparent to one skilled in the art in view of the teachings herein.

This larger torque requirement to rotate threaded body in the second angular direction may inhibit accidental rotation of threaded body (366) after yoke assembly (300) and the rest of instrument (100) of assembled. Therefore, locking teeth (370, 390) may help ensure that engagement spring (302) does not accidentally and undesirably lengthen after assembling instrument (100).

While in the current example, locking teeth (370, 390) are angled in order to inhibit engagement spring (302) from accidentally and undesirably lengthening, locking teeth (370, 390) may also be have sloped surface (372, 374, 392, 394) forming additional/alternative sloped angles to achieve other desirable functions. For example, FIGS. 12A and 13A show threaded adjustable member (360) and adjustable spring seat (380), respectively, with alternative locking teeth (970, 990) in replacement of locking teeth (370, 390) described above. As will be described in greater detail below, alternative locking teeth (970, 990) includes gradual sloped surfaces (972, 992) that additionally extend along respective planes to define vertically opposite sloped angles (α1, α2) in the radial direction (R). In particular, locking teeth (970, 990) are configured to contact each other in order to help align threaded adjustable member (360) and adjustable spring seat (380) along an axis coaxial or parallel with the longitudinal axis (LA) defined by shaft assembly (140).

Gradually sloped surfaces (972, 992) and inclined sloped surfaces (974, 994) are substantially similar to gradually sloped surfaces (392, 392) and inclined sloped surfaces (374, 394) described above, but with differences described herein. In particular, sloped surfaces (972, 992) extend to define a sloped angle (α1, α2) relative to the radial direction (R). Sloped surfaces (972) of locking teeth (370) define a sloped angle (al) relative to the radial direction (R) that is vertically opposite with respective sloped angle (α2) of sloped surfaces (992) of locking teeth (390). Therefore, when assembled, surfaces (972, 992) are substantially flush with each other. Due to engagement spring (302) biasing locking teeth (970, 990) into engagement with each other, the geometry of sloped surfaces (972, 992) may help threaded adjustable member (360) and adjustable spring seat (380) align with each other along longitudinal axis (LA) defined by shaft assembly (140). In other words, due to sloped surfaces (972, 992) extending along vertically opposite angles (α1, α2) with respect to the radial direction (R), locking teeth (970, 990) may engage each other to force threaded adjustable member (360) and adjustable spring seat (380) into longitudinal alignment.

While in the current example, gradually sloped surfaces (972, 992) define angles (α1, α2) with respect to the radial direction (R), inclined sloped surfaces (974, 994) may also define angles with respect to the radial direction (R) in order to longitudinally align threaded adjustable member (360) and adjustable spring seat (380).

Additionally, in some examples, locking teeth (370, 390, 970, 990) may be entirely omitted. In such instances threaded body (360) of threaded adjustable member (360) may have self-locking threads in order to prevent threaded body (360) from accidentally rotating in the second angular direction. Therefore, the self-locking threads may help ensure that engagement spring (302) does not accidentally and undesirably lengthen after assembling instrument (100).

B. Second Exemplary Yoke Assembly with Adjustment Feature

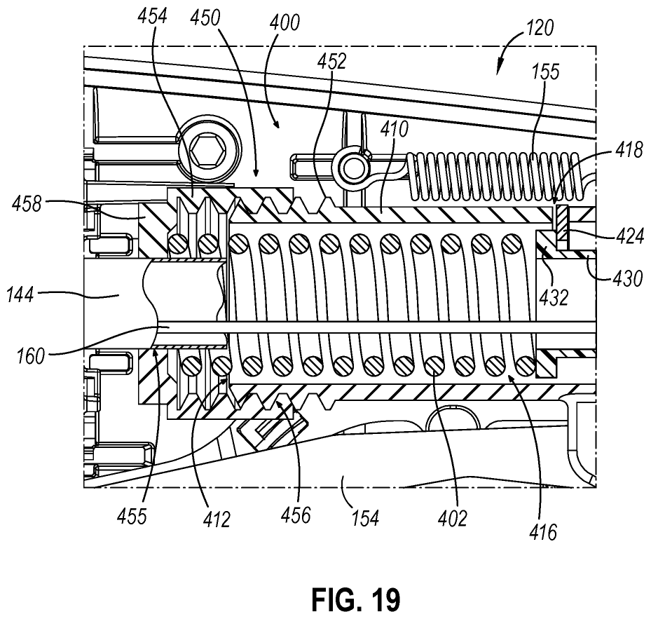

FIG. 19 shows an exemplary yoke assembly (400) that may be readily incorporated into instrument (100) in replacement of yoke assembly (200) described above. Yoke assembly (400) is substantially similar to yoke assembly (200) described above, with differences elaborated below. In particular, yoke assembly (400) includes an adjustment assembly (450) configured to adjust or tune the length of an engagement spring (402) after elongated jaw closure member (160) is fixed to a hollow pull tube (not shown).

Therefore, yoke assembly (400) includes engagement spring (402), hollow pull tube (not shown), a rotating connecting body (not shown), a translating ring (not shown), a yoke housing (410), a first clip (424), a second clip (not shown), a spring engagement collar (430) having a spring side flange (432), and a proximal collar (not shown); which are substantially similar to engagement spring (202), hollow pull tube (204), rotating connecting body (206), translating ring (208), yoke housing (210), first clip (224), second clip (226), spring engagement collar (230), and proximal collar (240), respectively, with differences elaborated below.

Yoke housing (410) defines an open distal end (412), an open proximal end (not shown), a cavity (416), and clip slots (418, not shown); which are substantially similar to open distal end (212), open proximal end (214), cavity (216), and clip slots (218, 220), respectively described above, with difference elaborated below.

In this example, adjustment assembly (450) includes outer diameter threading (452) on yoke housing (410) and a distal adjustment cap (454). As will be described in greater detail below, distal adjustment cap (454) is configured to rotate relative to outer diameter threading (452) in order to selectively adjust/tune the spring length (and therefore the preload force) of engagement spring (402) during assembly of instrument (100). Therefore, an operator or manufacturing machine may change the amount of proximal force engagement spring (402) imparts on collar (430) without undesirably compressing spring (402) during exemplary closure of jaws (182, 184) in accordance with the description herein.

Outer diameter threading (452) is located on the outer diameter and the distal end of yoke housing (410). Distal adjustment cap (454) defines an interior portion having inner diameter threading (456) and a proximal face (458). Inner diameter threading (456) is configured to suitably engage outer diameter threading (452) such that rotation of cap (454) relative to yoke housing (410) causes cap (452) to acuate longitudinally relative to yoke housing (410). Proximal face (458) abuts against a distal end of engagement spring (402) such that as cap actuates longitudinally relative to yoke housing (410), engagement spring (402) changes spring length.

Therefore, an operator or manufacturing machine may rotate cap (454) in a first rotational direction in order to drive proximal face (458) proximally toward cavity (416) in order to compress engagement spring (402). With a shorter spring length, engagement spring (402) may impart a greater proximal reactionary force against spring engagement collar (430) without overly compressing during elementary closing of jaws (182, 184). In other words, adjustment assembly (450) may be utilized to ensure closure of trigger (126) results in a suitable closure force between jaws (182, 184).

Conversely, an operator or manufacturing machine may rotate cap (454) in a second, opposite, rotational direction in order to drive proximal face (458) distally away from cavity (416) in order to expand engagement spring (402).

C. Third Exemplary Yoke Assembly with Adjustment Feature

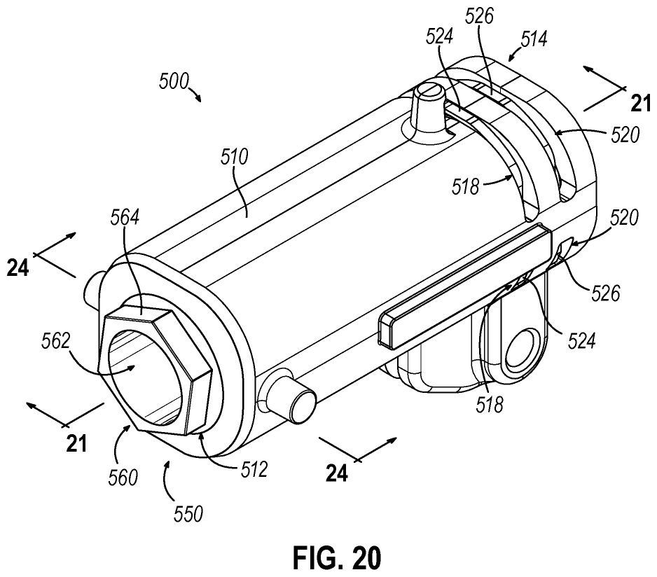

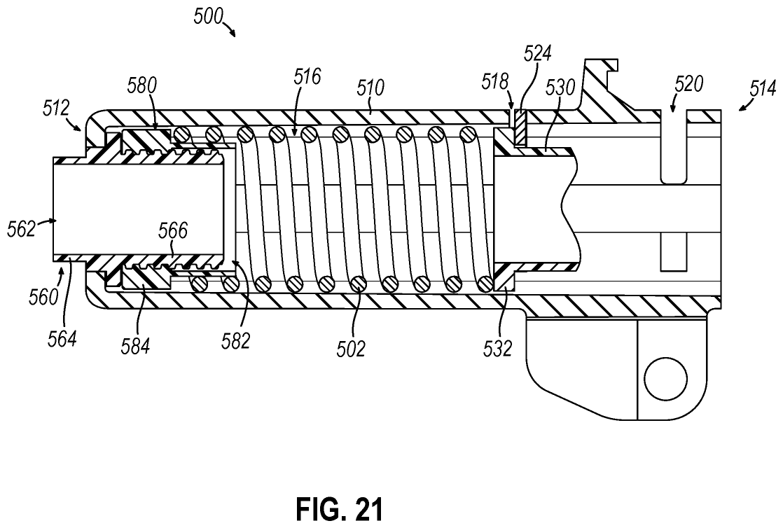

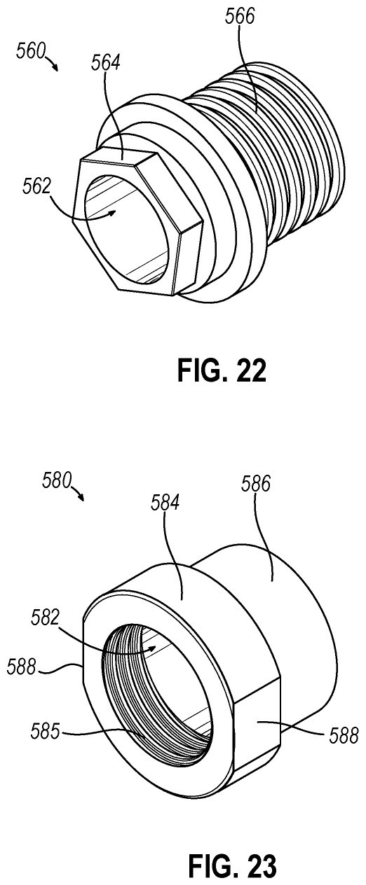

FIG. 20-24 shows an exemplary yoke assembly (500) that may be readily incorporated into instrument (100) in replacement of yoke assembly (200) described above. Yoke assembly (500) is substantially similar to yoke assembly (200) described above, with differences elaborated below. In particular, yoke assembly (500) includes an adjustment assembly (550) configured to adjust or tune the length of an engagement spring (502) after elongated jaw closure member (160) is fixed to a hollow pull tube (not shown).

Therefore, yoke assembly (500) includes engagement spring (502), hollow pull tube (not shown), a rotating connecting body (not shown), a translating ring (not shown), a yoke housing (510), a first clip (524), a second clip (not shown), a spring engagement collar (530) having a spring side flange (532), and a proximal collar (not shown); which are substantially similar to engagement spring (202), hollow pull tube (204), rotating connecting body (206), translating ring (208), yoke housing (210), first clip (224), second clip (226), spring engagement collar (230), and proximal collar (240), respectively, with differences elaborated below.

Yoke housing (510) defines an open distal end (512), an open proximal end (514), a cavity (516), and clip slots (518, 520); which are substantially similar to open distal end (212), open proximal end (214), cavity (216), and clip slots (218, 220), respectively described above, with difference elaborated below.

In this example, adjustment assembly (550) includes a rotating body (560) and a translating nut (580). As will be described in greater detail below, rotating body (560) is configured to rotate in the confines of yoke housing (510) in order to drive translation of translating nut (580) within cavity (516) to selectively adjust/tune the spring length (the therefore the preload force) of engagement spring (502) during assembly of instrument (100). Therefore, an operator or manufacturing machine may change the amount of proximal force engagement spring (502) imparts on collar (530) without overly compressing during exemplary closure of jaws (182, 184) in accordance with the description herein.

Rotating body (560) is rotationally disposed within open distal end (512) of yoke housing (510) such that rotating body (560) may rotate about its own longitudinal axis, but such that rotating body (560) is longitudinally fixed relative to yoke housing (512). Rotating body (560) includes a head (564) and a threaded body (566). Rotating body (560) also defines a through hole (562) extending from a proximal end to a distal end of rotating body (560). Through hole (562) is dimensioned to receive selective portions of shaft assembly (140). Head (564) may be engaged to rotate threaded body (566).

Translating nut (580) includes a spring engagement flange (584) and an interior sleeve (586). Spring engagement flange (584) has inner diameter threading (585) and a pair of flats (588). Translating nut (580) also defines a through hole (582) extending from a proximal end to a distal end. Through hole (582) is dimensioned to receive selective portions of shaft assembly (140).

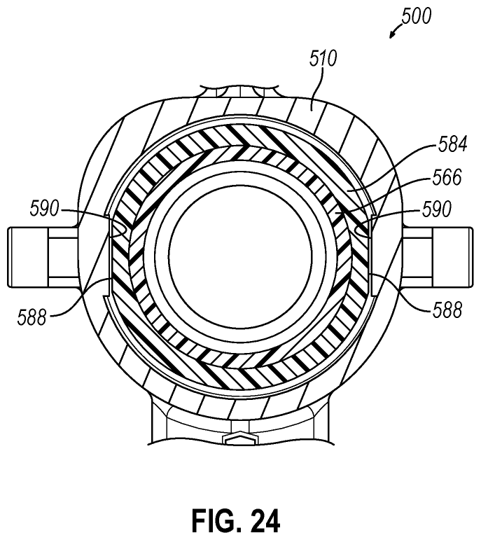

Inner diameter threading (585) is dimensioned to suitably mesh with threaded body (566). Additionally, translating nut (580) is rotationally fixed relative to yoke housing (510) by interaction between flats (588) of spring engagement flange (584) and flats (590) (see FIG. 24) of yoke housing (510). Therefore, rotation of threaded body (566) about its own longitudinal axis is configured to drive translation of nut (580) within cavity (516) of housing (510).

Spring engagement flange (584) abuts against a distal end of engagement spring (502) such that as nut (580) actuates longitudinally relative to yoke housing (510), engagement spring (502) changes its spring length.

Therefore, an operator or manufacturing machine may rotate body (560) in a first rotational direction in order to drive nut (580) proximally into cavity (516) in order to compress engagement spring (502). With a shorter spring length, engagement spring (502) may impart a greater proximal reactionary force against spring engagement collar (530) without overly compressing during elementary closing of jaws (182, 184). In other words, adjustment assembly (550) may be utilized to ensure closure of trigger (126) results in a suitable closure force between jaws (182, 184).

Conversely, an operator or manufacturing machine may rotate body (560) in a second, opposite, rotational direction in order to drive nut (580) distally within cavity (516) in order to expand engagement spring (502).

D. Fourth Exemplary Yoke Assembly with Adjustment Feature

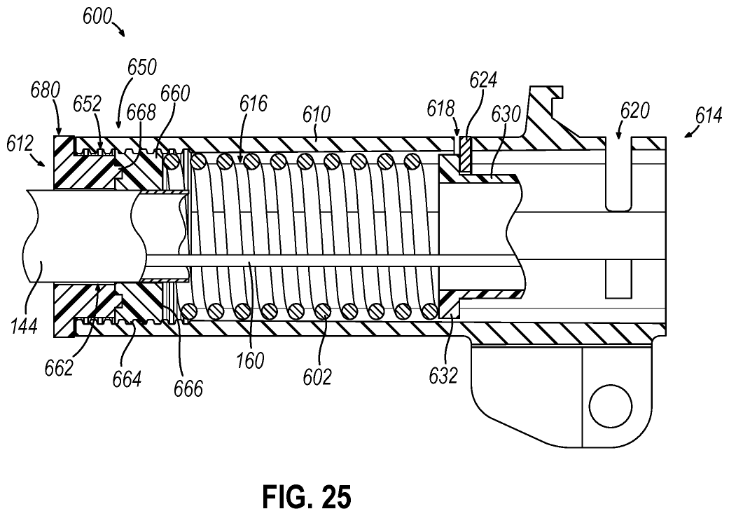

FIG. 25 shows an exemplary yoke assembly (600) that may be readily incorporated into instrument (100) in replacement of yoke assembly (200) described above. Yoke assembly (600) is substantially similar to yoke assembly (200) described above, with differences elaborated below. In particular, yoke assembly (600) includes an adjustment assembly (650) configured to adjust or tune the length of an engagement spring (602) after elongated jaw closure member (160) is fixed to a hollow pull tube (not shown).

Therefore, yoke assembly (600) includes engagement spring (602), hollow pull tube (not shown), a rotating connecting body (not shown), a translating ring (not shown), a yoke housing (610), a first clip (624), a second clip (not shown), a spring engagement collar (630) having a spring side flange (632), and a proximal collar (not shown); which are substantially similar to engagement spring (202), hollow pull tube (204), rotating connecting body (206), translating ring (208), yoke housing (210), first clip (224), second clip (226), spring engagement collar (230), and proximal collar (240), respectively, with differences elaborated below.

Yoke housing (610) defines an open distal end (612), an open proximal end (614), a cavity (616), and clip slots (618, 620); which are substantially similar to open distal end (212), open proximal end (214), cavity (216), and clip slots (218, 220), respectively described above, with difference elaborated below.

In this example adjustment assembly (650) includes an internal threading (652) on the interior surface of yoke housing (610), a compression ring (660), and a removable torqueing tool (680). Compression ring (660) includes a threading (664), a compression surface (666), and an engagement feature (668). Compression ring (660) defines a through hole (662) extending from a proximal end to a distal end of compression ring (660). Through hole (662) is dimensioned to receive selective portions of shaft assembly (140).

Threading (664) of compression ring (660) meshes with internal threading (652) of yoke housing (610) such that rotation of compression ring (660) relative to yoke housing (610) drives longitudinal actuation of compression ring (660). Compression surface (666) abuts against a distal end of engagement spring (602) such that as compression ring (660) actuates longitudinally relative to yoke housing (610), engagement spring (602) changes its spring length (and therefore the preload force).

Removable torqueing tool (680) is configured to selectively couple with engagement features (668) of compression ring (660) in order to drive rotation of compression ring (660) relative to yoke housing (610), thereby driving longitudinal actuation of compression ring (660) relative to yoke housing (610).

Therefore, an operator or manufacturing machine may selectively couple removable torqueing tool (680) with compression ring (660) in order to rotate ring (660) in a first rotational direction. Removable torqueing tool (680) may drive compression surface (666) proximally within cavity (616) in order to compress engagement spring (602). With a shorter spring length, engagement spring (602) may impart a greater proximal reactionary force against spring engagement collar (630) without overly compressing during elementary closing of jaws (182, 184). In other words, adjustment assembly (650) may be utilized to ensure closure of trigger (126) results in a suitable closure force between jaws (182, 184).

Conversely, an operator or manufacturing machine may rotate compression ring (660) in a second, opposite, rotational direction in order to drive compression surface (666) distally within cavity (616) in order to expand engagement spring (602).

E. Fifth Exemplary Yoke Assembly with Adjustment Feature

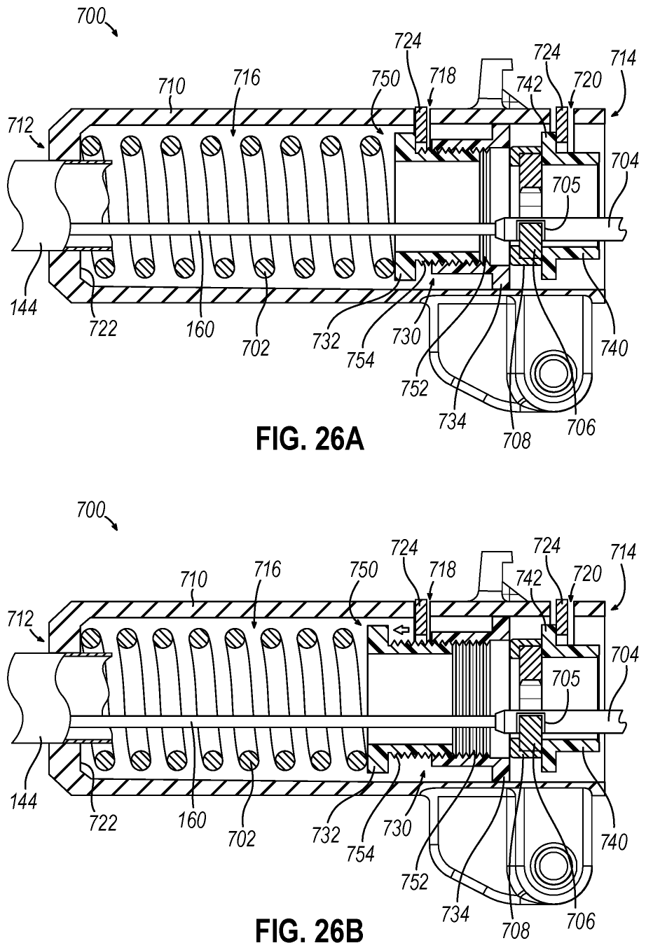

FIGS. 26A-26B show an exemplary yoke assembly (700) that may be readily incorporated into instrument (100) in replacement of yoke assembly (200) described above. Yoke assembly (700) is substantially similar to yoke assembly (200) described above, with differences elaborated below. In particular, yoke assembly (700) includes an adjustment assembly (750) configured to adjust or tune the length of an engagement spring (702) after elongated jaw closure member (160) is fixed to a hollow pull tube (04).

Therefore, yoke assembly (700) includes engagement spring (702), hollow pull tube (704) having flat surfaces (705), a rotating connecting body (706), a translating ring (708), a yoke housing (710), a first clip (724), a second clip (726), a spring engagement collar (730), and a proximal collar (740) having a flange (742); which are substantially similar to engagement spring (202), hollow pull tube (204) having flat surfaces (205), rotating connecting body (206), translating ring (208), yoke housing (210), first clip (224), second clip (226), spring engagement collar (230), and proximal collar (240) having flange (242), respectively, with differences elaborated below.

Yoke housing (710) includes a proximally facing interior surface (722) which is substantially similar to proximally facing interior surface (222) described above. Yoke housing (710) also defines an open distal end (712), an open proximal end (714), a cavity (716), and clip slots (718, 720); which are substantially similar to open distal end (212), open proximal end (214), cavity (216), and clip slots (218, 220), respectively described above, with difference elaborated below.

In this example, spring engagement collar (730) is split into two pieces in order to form adjustable assembly (750), such that adjustable assembly (750) is configured to compress spring (702) from a proximal end thereof. In particular, the portion of spring engagement collar (730) having spring side flange (732) includes an outer diameter threading portion (754), while the portion of spring engagement collar (730) having second flange (734) includes an inner diameter threading portion (752). Threading portions (752, 754) mesh with each other such that if spring side flange (732) rotates relative to second flange (734), the overall length of spring engagement collar (730) changes. Therefore, an operator or manufacturing machine may rotate spring side flange (732) relative to second flange (734) in a first rotational direction in order to drive spring side flange (732) distally, thereby compressing engagement spring. With a shorter spring length (and therefore a greater preload force), engagement spring (702) may impart a greater proximal reactionary force against spring engagement collar (730) without overly compressing during elementary closing of jaws (182, 184). In other words, adjustment assembly (750) may be utilized to ensure closure of trigger (126) results in a suitable closure force between jaws (182, 184).

F. Sixth Exemplary Yoke Assembly with Adjustment Feature

In some instances, it may be desirable to adjust/control the spring length of an engagement spring (and therefore adjust/control the preload force) during or immediately prior to a surgical procedure.

For instance, once instrument (100) is initially coupled to power source (not shown) via power cable (10) in order to suitably use instrument (100), it may be desirable for instrument (100) to initiate a calibration process where the preload force of engagement spring (302) is measured and adjusted accordingly. As another example, during a surgical procedure, force sensor (195) (see FIGS. 5A-5C) may measure a closure force that is either too high or too low while jaws (182, 184) are in the fully closed configuration, resulting in the undesirable consequences mentioned above. As another example, the desired preload force provided by engagement spring (302) may deviate during real time use. For instance, the desired preload force provided by engagement spring (302) may deviate in response to end effector (180) being in various articulated configurations. Engagement spring (302) may require a first preload force while end effector (180) is in a non-articulated configuration, and may also require a second preload force while end effector (180) is in an articulated configuration. Therefore, it may be desirable for instrument (100) to calibrate the preload force of engagement spring (302) to suitably adjust the closure force provided by jaws (182, 184) in real time.

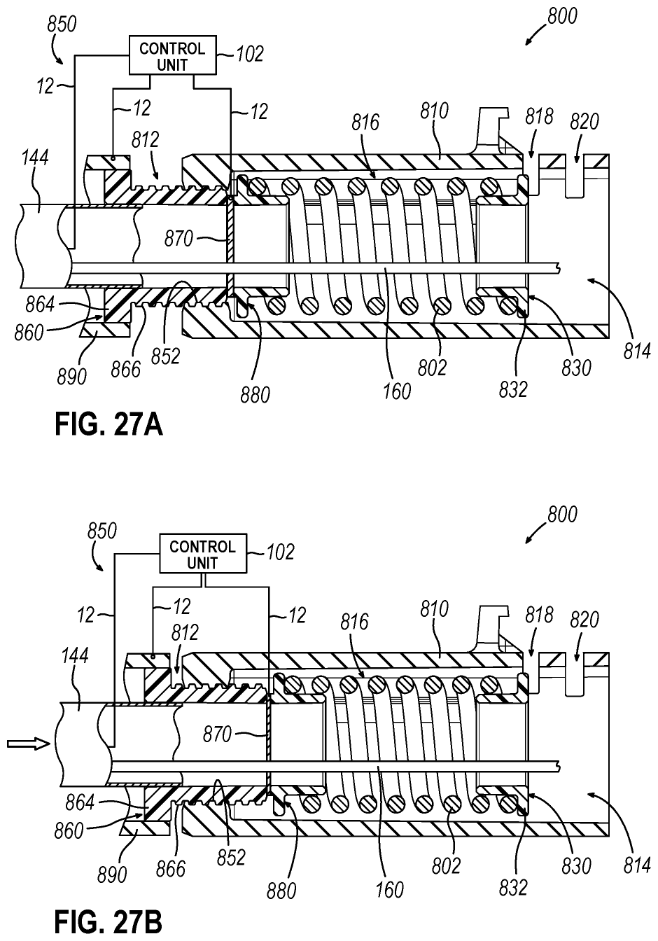

FIGS. 27A-27B show an exemplary yoke assembly (800) that may be readily incorporated into instrument (100) in replacement of yoke assembly (200) described above. Yoke assembly (800) is substantially similar to yoke assembly (200) described above, with differences elaborated below. In particular, yoke assembly (800) includes an adjustment assembly (850) configured to adjust or tune the length of an engagement spring (802) prior to, or during, exemplary use of instrument (100).

Therefore, yoke assembly (800) includes engagement spring (802), hollow pull tube (not shown), a rotating connecting body (not shown), a translating ring (not shown), a yoke housing (810), a first clip (not shown), a second clip (not shown), a spring engagement collar (830) having a spring side flange (832), and a proximal collar (not shown); which are substantially similar to engagement spring (202), hollow pull tube (204), rotating connecting body (206), translating ring (208), yoke housing (210), first clip (224), second clip (226), spring engagement collar (230), and proximal collar (240), respectively, with differences elaborated below.

Yoke housing (810) defines an open distal end (812), an open proximal end (814), a cavity (816), and clip slots (818, 820); which are substantially similar to open distal end (212), open proximal end (214), cavity (216), and clip slots (218, 220), respectively described above, with difference elaborated below.

In this example, adjustment assembly (850) includes an adjustable threaded member (860), an adjustable spring seat (880), a force measuring device such as a load cell (870) interposed between adjustable threaded member (860) and adjustable spring seat (880), and a motorized gear assembly (890) operatively attached to adjustable threaded member (860). As will be described in greater detail below, load cell (870) is configured to measure the preload force provided by engagement spring (802); while motorized gear assembly (890) is configured to adjust the length of engagement spring (802) based on information provided by load cell (870), force sensor (195) (see FIGS. 5A-5C), and/or signals provided control unit in response to any other suitable conditions as would be apparent to one skilled in the art in view of the teachings herein.

Adjustable threaded member (860) and adjustable spring seat (880) may be substantially similar to adjustable threaded member (360) and adjustable spring seat (380) described above, with differences elaborated below. Therefore, adjustable threaded member (860) includes a head (864), and a threaded body (866), which may be substantially similar to head (364) and threaded body (366) described above, with differences elaborated below. Threaded body (866) is operatively engaged with a female housing (852) of yoke housing (810) in similar fashion to threaded body (366) and female threading (352) described above.

Motorized gear assembly (890) is configured to rotate threaded member (860) relative to yoke housing (810) in order to selectively adjust the longitudinal position of threaded member (860) relative to yoke housing (810). Motorized gear assembly (890) is in electrical communication with control unit (102) via communication wire (12) such that control unit (102) may instruct motorized gear assembly (890) when to rotate threaded member (860), and by how much to rotate threaded member (860). Therefore, control unit (102) is configured to selectively adjust the spring length (and therefore the preload force) of engagement spring (802) via motorized gear assembly (890).

Motorized gear assembly (890) may include any suitable components as would be apparent to one skilled in the art in view of the teachings herein. For instance, motorized gear assembly (890) may include a motor, gear box, drive shaft, and coupling body. Additionally, motorized gear assembly (890) may be configured to longitudinally actuate with threaded member (860) relative to yoke housing (810). Alternative, motorized gear assembly (890) may be configured to rotate relative to yoke housing (810) in order to longitudinally actuate threaded member (860) while (A) remaining longitudinally fixed relative to yoke housing (810) and (B) remaining operatively coupled with threaded member (860).

As mentioned above, load cell (870) is interposed between adjustable threaded member (860) and adjustable spring seat (880). Load cell (870) may be operatively attached to either adjustable threaded member (860), or adjustable spring seat (880). Additionally, load cell (870) is in communication with control unit (102) via communication wire (12).

Load cell (870) is configured to measure the compressive forces generated by engagement spring (802) and communicate that value to control unit (102). The force generated by engagement spring (802) and transmitted to load cell (870) via compression between spring seat (880) and adjustable threaded member (860) may be indicative of the preload force engagement spring (802) can impart on spring engagement collar (830) during exemplary closing of jaws (182, 184) in accordance with the description herein.

Control unit (102) may compare the compressive forces measured by load cell (870), which may be indicative of the preload force generated by engagement spring (802), and compare that measured value to a reference value associated with engagement spring (802) closing jaws (182, 184) with the desired closure force in accordance with the description herein. As mentioned above, force sensor (195) (see FIGS. 5A-5C) may also be in communication with control unit (102) via communication wire (12). Therefore, control unit (102) may calculate the reference value based on real time feedback from force sensor (195) (see FIGS. 5A-5C) as jaws (182, 184) are pivoted into the closed configuration. Alternatively, the reference value may be stored on control unit (102) prior to real time use of instruments (100). The reference value associated with a suitable preload force provided by engagement spring (802) may be determined by any suitable means as would be apparent to one skilled in the art in view of the teachings herein. Additionally, the reference value may change based on the articulated configuration of end effector (180) such that control unit (102) may instruct motorized gear assembly (890) to change the length of engagement spring (802) based, at least in part, by the degree at which end effector (180) is articulated. This functionality may help ensure the closure force provided by jaws (182, 184) is substantially consistent regardless if end effector (180) is in an articulated or non-articulated configuration.

If the compressive force measured by load cell (870) is too great (i.e., indicative of jaws (182, 184) over-compressing and potentially causing structural damage to the tissue and/or end effector (180)), control unit (102) may instruct motorized gear assembly (890) to rotate adjustable threaded member (860) in order to lengthen engagement spring (802) (and therefore reduce the preload force) until the compressive force measured by load cell (870) is suitably close to the reference value stored in control unit (102).

Conversely, if the compressive force measured by load cell (870) is too low (i.e., indicative of jaws electrode surfaces (194, 196) of jaws (182, 184) failing to apply a suitable degree of RF energy to the tissue, leading to poor welding or sealing to the tissue), control unit (102) may instruct motorized gear assembly (890) to rotate adjustable threaded member (860) in order to shorten engagement spring (802) (and therefore increase the preload force) until the compressive force measured by load cell (870) is suitably close to the reference value stored in control unit (102).

Control unit (102) may also be configured to instruct motorized gear assembly (890) based solely on information received from force sensor (195) (see FIGS. 5A-5C), rather than load cell (870). Therefore, in some instances, load cell (870) is optional. Conversely, control unit (102) may be configured to instruct motorized gear assembly (890) based solely on information received from load cell (870). Therefore, in some instances, force sensor (195) (see FIGS. 5A-5C) is optional. Alternatively, control unit (102) may be configured to instruct motorized gear assembly (890) based on a combination of information received from force sensor (195) (see FIGS. 5A-5C), load cell (870), and any other suitable sensors and measuring devices as would be apparent to one skilled in the art in view of the teachings herein.

Control unit (102) may instruct motorized gear assembly (890) in accordance with the description herein during any suitable moment as would be apparent to one skilled in the art in view of the teachings herein. For instance, once power cable (10) is initially plugged into a power source, control unit (102) may automatically run through a calibration cycle in order to ensure engagement spring (802) imparts a suitable preload force. Additionally, or alternatively, control unit (102) may instruct motorized gear assembly (890) to suitable shorten/lengthen engagement spring (802) during real time use of instrument (100).

In some instances, yoke assembly (800), adjustment assembly (850), and shaft assembly (140) may be a modular attachment, such that shaft assembly (140), yoke assembly (800), and adjustment assembly (850) may be configured to selectively attach and detach from handle assembly (120). In some instances, where shaft assembly (140), yoke assembly (800), and adjustment assembly (850) are a modular attachment, such a modular attachment may be configured to operatively attach with a robotic interface such that end effector (180) may be suitably controlled via a robotic device.

In such instances, control unit (102) of handle assembly (120)/robotic interface may be configured to selectively establish communication with force sensor (195), load cell (870), and motorized gear assembly (890) once the module attachment is suitably coupled with handle assembly (120)/robotic interface. In such instances, selective communication with control unit (102) may be formed through any suitable means as would be apparent to one skilled in the art in view of the teachings herein. For instance, slip rings may be used to selectively establish communication between control unit (102) and electrical components of the modular attachment. Additionally, the module attachment may contain its own control unit containing suitable information specific to that modular attachment (such as desired preload force, spring length, desired distal tip force measured by force sensor (195), etc.).

IV. Exemplary Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) an end effector, comprising: (i) a first jaw, and (ii) a second jaw, wherein the second jaw is configured to actuate relative to the first jaw between an open configuration and a closed configuration; (b) a shaft assembly extending proximally from the end effector, wherein the shaft assembly extends along a longitudinal axis; (c) a jaw closure assembly, wherein the jaw closure assembly comprises: (i) an elongated jaw closure member configured to actuate relative to the shaft assembly between a first position and a second position to drive the second jaw between the open configuration and the closed configuration, (ii) a coupling body attached to the elongated jaw closure member, (iii) a yoke housing configured to acuate the coupling body and the elongated jaw closure member relative to the shaft assembly between the first position and the second position, and (iv) an engagement spring disposed within the yoke housing, wherein the engagement spring is configured to bias the coupling body and the elongated jaw closure member into operative engagement with the yoke housing; and (d) an adjustment assembly configured to adjust a spring length of the engagement spring to ensure the coupling body and the elongated jaw closure member remain in operative engagement with the yoke housing.

### Example 2

The apparatus of any one or more of the preceding Examples, wherein the adjustment assembly comprises a threaded adjustable member comprising a threaded body, wherein the threaded body is rotatable relative to the yoke housing to adjust the spring length of the engagement spring.

### Example 3

The apparatus of any one or more of the preceding Examples, wherein the threaded adjustable member defines a through hole dimensioned to receive the shaft assembly.

### Example 4

The apparatus of any one or more of the preceding Examples, wherein the yoke housing defines a threaded opening operatively coupled with the threaded body, wherein the threaded body is configured to translate relative to the yoke housing.

### Example 5

The apparatus of any one or more of the preceding Examples, wherein the adjustment assembly further comprises an adjustable spring seat interposed between the threaded body and the engagement spring.

### Example 6

The apparatus of any one or more of the preceding Examples, wherein the adjustable spring seat comprises a first set of locking teeth, wherein the threaded body comprises a complementary set of locking teeth.

### Example 7

The apparatus of any one or more of the preceding Examples, wherein the first set of locking teeth and the complementary set of locking teeth are configured to provided tactile feedback in response to rotation of the threaded body relative to the adjustable spring seat.

### Example 8

The apparatus of any one or more of the preceding Examples, wherein the first set of locking teeth and the complementary set of locking teeth are configured to inhibit rotation of the threaded body relative to the yoke housing in a first rotational direction.

### Example 9

The apparatus of any one or more of the preceding Examples, wherein the first set of locking teeth and the complementary set of locking teeth are configured to align the adjustable spring seat and the threaded body relative to each other along the longitudinal axis.

### Example 10

The apparatus of any one or more of the preceding Examples, wherein the adjustable spring seat comprise an angular locking projection, wherein the yoke housing defines a longitudinal channel dimensioned to receive the angular locking projection.

### Example 11

The apparatus of any one or more of the preceding Examples, wherein the adjustment assembly comprises a distal threaded cap configured to rotate relative to the yoke housing.

### Example 12

The apparatus of any one or more of the preceding Examples, wherein the adjustment assembly comprises a threaded body longitudinally fixed with the yoke housing and an adjustable nut configured to translate relative to the yoke housing.

### Example 13

The apparatus of any one or more of the preceding Examples, wherein the end effector further comprises a knife member.

### Example 14

The apparatus of any one or more of the preceding Examples, wherein the adjustment assembly comprises a removable torque tool.

### Example 15

The apparatus of any one or more of the preceding Examples, wherein the end effector comprises a first electrode associated with the first jaw and a second electrode associated with the second jaw.

### Example 16

The apparatus of any one or more of the preceding Examples, further comprising a control unit, wherein the adjustment assembly comprises a motorized gear assembly, wherein the control unit is configured to selectively activate the motorized gear assembly.

### Example 17

The apparatus of any one or more of the preceding Examples, further comprising a load cell configured to measure a preload force of the engagement spring, wherein the control unit is configured to selectively activate the motorized gear assembly based on a measurement of the load cell.

### Example 18

The apparatus of any one or more of the preceding Examples, further comprising a force sensor associated with the first jaw, wherein the control unit is configured to selectively activate the motorized gear assembly based on a measurement of the force sensor.

### Example 19

An apparatus, comprising: (a) an end effector, comprising: (i) a first jaw comprising a first electrode surface, and (ii) a second jaw comprising a second electrode surface, wherein the second jaw is configured to actuate relative to the first jaw between an open configuration and a closed configuration; (b) a shaft assembly extending proximally from the end effector, wherein the shaft assembly extends along a longitudinal axis; (c) a jaw closure assembly, wherein the jaw closure assembly comprises: (i) an actuating jaw closure member configured to actuate relative to the shaft assembly between a first position and a second position to drive the second jaw between the open configuration and the closed configuration, (ii) a yoke housing configured to drive the actuating jaw closure member relative to the shaft assembly between the first position and the second position, and (iii) a spring disposed within the yoke housing, wherein the spring is configured to bias the actuating jaw closure member into an operative position relative to the yoke housing; and (d) an adjustment assembly configured to adjust a preload of the spring to ensure the actuating jaw closure member remains in the operative position.

### Example 20

An apparatus, comprising: (a) an end effector, comprising: (i) a first jaw, and (ii) a second jaw, wherein the second jaw is configured to actuate relative to the first jaw between an open configuration and a closed configuration; (b) a shaft assembly extending proximally from the end effector, wherein the shaft assembly extends along a longitudinal axis; (c) a jaw closure assembly, wherein the jaw closure assembly comprises: (i) an elongated jaw closure member configured to translate relative to the shaft assembly to drive the second jaw between the open configuration and the closed configuration, (ii) a yoke housing configured to acuate the elongated jaw closure member relative to the shaft assembly, and (iii) an engagement spring configured to bias the elongated jaw closure member into a fixed position relative to the yoke housing; and (d) an adjustment assembly configured to adjust a spring length of the engagement spring to ensure the elongated jaw closure member remains in the fixed position relative to the yoke housing.

### Example 21

A method of setting up a surgical instrument, the method comprising: (a) imparting a proximal force on an elongated jaw closure member to thereby actuate a second jaw relative to a first jaw into a closed configuration; (b) fixing a proximal portion of the elongated jaw closure member to a coupling body, wherein the coupling body is operatively coupled with a yoke assembly; and (c) compressing an engagement spring housed within a yoke assembly to thereby increase a proximal preload force on the coupling body.

### Example 22

The method of Example 21, further comprising actuating the engagement spring and the yoke assembly proximally to actuate the coupling body and the elongated jaw member proximally in order to acuate the second jaw into the closed configuration.

### Example 23

The method of any one or more of the preceding Examples, wherein the act of compressing the engagement spring within the yoke assembly is performed after fixing the proximal portion of the elongated jaw closure member to the coupling body.

### Example 24

The method of any one or more of the preceding Examples, wherein the yoke assembly comprises a threaded adjustable member, wherein compressing the engagement spring comprises rotating the threaded adjustable member relative to a yoke housing of the yoke assembly.

### Example 25

The method of any one or more of the preceding Examples, wherein the coupling body comprises a hollow pull tube, wherein the elongated jaw closure member is slidably disposed within the hollow pull tube during the act of imparting a proximal force on an elongated jaw closure member.

### Example 26

The method of any one or more of the preceding Examples, further comprising actuating the yoke assembly distally to actuate the second jaw into an open configuration.

### Example 27

The apparatus of any one or more of the preceding Examples, further comprising an articulation assembly interposed between the end effector and the shaft assembly.

### Example 28

The apparatus of any one or more of the preceding Examples, further comprising a proximal body, wherein the yoke housing is disposed within the proximal body.

### Example 29

The apparatus of any one or more of the preceding Examples, wherein the proximal body comprises a handle assembly.

IV. Miscellaneous

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) an end effector, comprising:
(i) a first jaw, and
(ii) a second jaw, wherein the second jaw is configured to actuate relative to the first jaw between an open configuration and a closed configuration;
(b) a shaft assembly extending proximally from the end effector, wherein the shaft assembly extends along a longitudinal axis;
(c) a jaw closure assembly, wherein the jaw closure assembly comprises:
(i) an elongated jaw closure member configured to actuate relative to the shaft assembly between a first position and a second position to drive the second jaw between the open configuration and the closed configuration,
(ii) a coupling body attached to the elongated jaw closure member,
(iii) a yoke housing configured to acuate the coupling body and the elongated jaw closure member relative to the shaft assembly between the first position and the second position, and
(iv) an engagement spring disposed within the yoke housing, wherein the engagement spring is configured to bias the coupling body and the elongated jaw closure member into operative engagement with the yoke housing; and
(d) an adjustment assembly configured to adjust a spring length of the engagement spring to ensure the coupling body and the elongated jaw closure member remain in operative engagement with the yoke housing.

2. The apparatus of claim 1, wherein the adjustment assembly comprises a threaded adjustable member comprising a threaded body, wherein the threaded body is rotatable relative to the yoke housing to adjust the spring length of the engagement spring.

3. The apparatus of claim 2, wherein the threaded adjustable member defines a through hole dimensioned to receive the shaft assembly.

4. The apparatus of claim 2, wherein the yoke housing defines a threaded opening operatively coupled with the threaded body, wherein the threaded body is configured to translate relative to the yoke housing.

5. The apparatus of claim 4, wherein the adjustment assembly further comprises an adjustable spring seat interposed between the threaded body and the engagement spring.

6. The apparatus of claim 5, wherein the adjustable spring seat comprises a first set of locking teeth, wherein the threaded body comprises a complementary set of locking teeth.

7. The apparatus of claim 6, wherein the first set of locking teeth and the complementary set of locking teeth are configured to provided tactile feedback in response to rotation of the threaded body relative to the adjustable spring seat.

8. The apparatus of claim 6, wherein the first set of locking teeth and the complementary set of locking teeth are configured to inhibit rotation of the threaded body relative to the yoke housing in a first rotational direction.

9. The apparatus of claim 6, wherein the first set of locking teeth and the complementary set of locking teeth are configured to align the adjustable spring seat and the threaded body relative to each other along the longitudinal axis.

10. The apparatus of claim 6, wherein the adjustable spring seat comprise an angular locking projection, wherein the yoke housing defines a longitudinal channel dimensioned to receive the angular locking projection.

11. The apparatus of claim 1, wherein the adjustment assembly comprises a distal threaded cap configured to rotate relative to the yoke housing.

12. The apparatus of claim 1, wherein the adjustment assembly comprises a threaded body longitudinally fixed with the yoke housing and an adjustable nut configured to translate relative to the yoke housing.

13. The apparatus of claim 1, wherein the end effector further comprises a knife member.

14. The apparatus of claim 1, wherein the adjustment assembly comprises a removable torque tool.

15. The apparatus of claim 1, wherein the end effector comprises a first electrode associated with the first jaw and a second electrode associated with the second jaw.

16. The apparatus of claim 1, further comprising a control unit, wherein the adjustment assembly comprises a motorized gear assembly, wherein the control unit is configured to selectively activate the motorized gear assembly.

17. The apparatus of claim 16, further comprising a load cell configured to measure a preload force of the engagement spring, wherein the control unit is configured to selectively activate the motorized gear assembly based on a measurement of the load cell.

18. The apparatus of claim 16, further comprising a force sensor associated with the first jaw, wherein the control unit is configured to selectively activate the motorized gear assembly based on a measurement of the force sensor.

19. An apparatus, comprising:
(a) an end effector, comprising:
(i) a first jaw comprising a first electrode surface, and
(ii) a second jaw comprising a second electrode surface, wherein the second jaw is configured to actuate relative to the first jaw between an open configuration and a closed configuration;
(b) a shaft assembly extending proximally from the end effector, wherein the shaft assembly extends along a longitudinal axis;
(c) a jaw closure assembly, wherein the jaw closure assembly comprises:
(i) an actuating jaw closure member configured to actuate relative to the shaft assembly between a first position and a second position to drive the second jaw between the open configuration and the closed configuration,
(ii) a yoke housing configured to drive the actuating jaw closure member relative to the shaft assembly between the first position and the second position, and
(iii) a spring disposed within the yoke housing, wherein the spring is configured to bias the actuating jaw closure member into an operative position relative to the yoke housing; and
(d) an adjustment assembly configured to adjust a preload of the spring to ensure the actuating jaw closure member remains in the operative position.

20. An apparatus, comprising:
(a) an end effector, comprising:
(i) a first jaw, and
(ii) a second jaw, wherein the second jaw is configured to actuate relative to the first jaw between an open configuration and a closed configuration;
(b) a shaft assembly extending proximally from the end effector, wherein the shaft assembly extends along a longitudinal axis;
(c) a jaw closure assembly, wherein the jaw closure assembly comprises:
(i) an elongated jaw closure member configured to translate relative to the shaft assembly to drive the second jaw between the open configuration and the closed configuration,
(ii) a yoke housing configured to acuate the elongated jaw closure member relative to the shaft assembly, and
(iii) an engagement spring configured to bias the elongated jaw closure member into a fixed position relative to the yoke housing; and
(d) an adjustment assembly configured to adjust a spring length of the engagement spring to ensure the elongated jaw closure member remains in the fixed position relative to the yoke housing.
